SEQUENCE LISTING (49 sequences)

SEQ ID NO: 1 - DNA, 40 bp, synthetic construct
  source 1..40: mol_type = other DNA; note = Nucleic acid of P1-1
  modified_base 1: mod_base = OTHER; note = NH2
ctacacgacgctcttccgatctgtcacataactggcctgc

SEQ ID NO: 2 - DNA, 40 bp, synthetic construct
  source 1..40: mol_type = other DNA; note = Nucleic acid of P1-2
  modified_base 1: mod_base = OTHER; note = NH2
ctacacgacgctcttccgatctcacgccggactggcctgc

SEQ ID NO: 3 - DNA, 40 bp, synthetic construct
  source 1..40: mol_type = other DNA; note = Nucleic acid of P1-3
  modified_base 1: mod_base = OTHER; note = NH2
ctacacgacgctcttccgatctggtccaggactggcctgc

SEQ ID NO: 4 - DNA, 40 bp, synthetic construct
  source 1..40: mol_type = other DNA; note = Nucleic acid of P1-4
  modified_base 1: mod_base = OTHER; note = NH2
ctacacgacgctcttccgatctatcgaatgactggcctgc

SEQ ID NO: 5 - DNA, 40 bp, synthetic construct
  source 1..40: mol_type = other DNA; note = Nucleic acid of P1-5
  modified_base 1: mod_base = OTHER; note = NH2
ctacacgacgctcttccgatctctagtataactggcctgc

SEQ ID NO: 6 - DNA, 40 bp, synthetic construct
  source 1..40: mol_type = other DNA; note = Nucleic acid of P1-6
  modified_base 1: mod_base = OTHER; note = NH2
ctacacgacgctcttccgatctatggctaaactggcctgc

SEQ ID NO: 7 - DNA, 40 bp, synthetic construct
  source 1..40: mol_type = other DNA; note = Nucleic acid of P1-7
  modified_base 1: mod_base = OTHER; note = NH2
ctacacgacgctcttccgatctagacatgcactggcctgc

SEQ ID NO: 8 - DNA, 40 bp, synthetic construct
  source 1..40: mol_type = other DNA; note = Nucleic acid of P1-8
  modified_base 1: mod_base = OTHER; note = NH2
ctacacgacgctcttccgatctaggcgatcactggcctgc

SEQ ID NO: 9 - DNA, 40 bp, synthetic construct
  source 1..40: mol_type = other DNA; note = Nucleic acid of P1-9
  modified_base 1: mod_base = OTHER; note = NH2
ctacacgacgctcttccgatcttgtccttaactggcctgc

SEQ ID NO: 10 - DNA, 40 bp, synthetic construct
  source 1..40: mol_type = other DNA; note = Nucleic acid of P1-10
  modified_base 1: mod_base = OTHER; note = NH2
ctacacgacgctcttccgatctgattaggaactggcctgc

SEQ ID NO: 11 - DNA, 40 bp, synthetic construct
  source 1..40: mol_type = other DNA; note = Nucleic acid of P1-11
  modified_base 1: mod_base = OTHER; note = NH2
ctacacgacgctcttccgatcttggatccaactggcctgc

SEQ ID NO: 12 - DNA, 40 bp, synthetic construct
  source 1..40: mol_type = other DNA; note = Nucleic acid of P1-12
  modified_base 1: mod_base = OTHER; note = NH2
ctacacgacgctcttccgatctcataagcgactggcctgc

SEQ ID NO: 13 - DNA, 40 bp, synthetic construct
  source 1..40: mol_type = other DNA; note = Nucleic acid of P1-13
  modified_base 1: mod_base = OTHER; note = NH2
ctacacgacgctcttccgatctactgtattactggcctgc

SEQ ID NO: 14 - DNA, 40 bp, synthetic construct
  source 1..40: mol_type = other DNA; note = Nucleic acid of P1-14
  modified_base 1: mod_base = OTHER; note = NH2
ctacacgacgctcttccgatctccttgcggactggcctgc

SEQ ID NO: 15 - DNA, 40 bp, synthetic construct
  source 1..40: mol_type = other DNA; note = Nucleic acid of P1-15
  modified_base 1: mod_base = OTHER; note = NH2
ctacacgacgctcttccgatctaggtgtagactggcctgc

SEQ ID NO: 16 - DNA, 40 bp, synthetic construct
  source 1..40: mol_type = other DNA; note = Nucleic acid of P1-16
  modified_base 1: mod_base = OTHER; note = NH2
ctacacgacgctcttccgatctggagattaactggcctgc

SEQ ID NO: 17 - DNA, 40 bp, synthetic construct
  source 1..40: mol_type = other DNA; note = Nucleic acid of P1-17
  modified_base 1: mod_base = OTHER; note = NH2
ctacacgacgctcttccgatctcgattacaactggcctgc

SEQ ID NO: 18 - DNA, 40 bp, synthetic construct
  source 1..40: mol_type = other DNA; note = Nucleic acid of P1-18
  modified_base 1: mod_base = OTHER; note = NH2
ctacacgacgctcttccgatctccggatagactggcctgc

SEQ ID NO: 19 - DNA, 40 bp, synthetic construct
  source 1..40: mol_type = other DNA; note = Nucleic acid of P1-19
  modified_base 1: mod_base = OTHER; note = NH2
ctacacgacgctcttccgatctcacttggaactggcctgc

SEQ ID NO: 20 - DNA, 40 bp, synthetic construct
  source 1..40: mol_type = other DNA; note = Nucleic acid of P1-20
  modified_base 1: mod_base = OTHER; note = NH2
ctacacgacgctcttccgatctgagaagttactggcctgc

SEQ ID NO: 21 - DNA, 46 bp, synthetic construct
  source 1..46: mol_type = other DNA; note = Nucleic acid of P2-1
nbaaaaaaaaaaaaaaaaaannnnnnnnatattgtggcaggccagt

SEQ ID NO: 22 - DNA, 46 bp, synthetic construct
  source 1..46: mol_type = other DNA; note = Nucleic acid of P2-2
nbaaaaaaaaaaaaaaaaaannnnnnnnctaggtgtgcaggccagt

SEQ ID NO: 23 - DNA, 46 bp, synthetic construct
  source 1..46: mol_type = other DNA; note = Nucleic acid of P2-3
nbaaaaaaaaaaaaaaaaaannnnnnnnttgcgttcgcaggccagt

SEQ ID NO: 24 - DNA, 46 bp, synthetic construct
  source 1..46: mol_type = other DNA; note = Nucleic acid of P2-4
nbaaaaaaaaaaaaaaaaaannnnnnnngtacgactgcaggccagt

SEQ ID NO: 25 - DNA, 46 bp, synthetic construct
  source 1..46: mol_type = other DNA; note = Nucleic acid of P2-5
nbaaaaaaaaaaaaaaaaaannnnnnnnctgtatttgcaggccagt

SEQ ID NO: 26 - DNA, 46 bp, synthetic construct
  source 1..46: mol_type = other DNA; note = Nucleic acid of P2-6
nbaaaaaaaaaaaaaaaaaannnnnnnntctgcgccgcaggccagt

SEQ ID NO: 27 - DNA, 46 bp, synthetic construct
  source 1..46: mol_type = other DNA; note = Nucleic acid of P2-7
nbaaaaaaaaaaaaaaaaaannnnnnnncgatcattgcaggccagt

SEQ ID NO: 28 - DNA, 46 bp, synthetic construct
  source 1..46: mol_type = other DNA; note = Nucleic acid of P2-8
nbaaaaaaaaaaaaaaaaaannnnnnnnttctcttggcaggccagt

SEQ ID NO: 29 - DNA, 46 bp, synthetic construct
  source 1..46: mol_type = other DNA; note = Nucleic acid of P2-9
nbaaaaaaaaaaaaaaaaaannnnnnnntagagatcgcaggccagt

SEQ ID NO: 30 - DNA, 46 bp, synthetic construct
  source 1..46: mol_type = other DNA; note = Nucleic acid of P2-10
nbaaaaaaaaaaaaaaaaaannnnnnnncgcgtgttgcaggccagt

SEQ ID NO: 31 - DNA, 46 bp, synthetic construct
  source 1..46: mol_type = other DNA; note = Nucleic acid of P2-11
nbaaaaaaaaaaaaaaaaaannnnnnnntaactaccgcaggccagt

SEQ ID NO: 32 - DNA, 46 bp, synthetic construct
  source 1..46: mol_type = other DNA; note = Nucleic acid of P2-12
nbaaaaaaaaaaaaaaaaaannnnnnnnccctcctggcaggccagt

SEQ ID NO: 33 - DNA, 46 bp, synthetic construct
  source 1..46: mol_type = other DNA; note = Nucleic acid of P2-13
nbaaaaaaaaaaaaaaaaaannnnnnnntaagcggagcaggccagt

SEQ ID NO: 34 - DNA, 46 bp, synthetic construct
  source 1..46: mol_type = other DNA; note = Nucleic acid of P2-14
nbaaaaaaaaaaaaaaaaaannnnnnnntactagcagcaggccagt

SEQ ID NO: 35 - DNA, 46 bp, synthetic construct
  source 1..46: mol_type = other DNA; note = Nucleic acid of P2-15
nbaaaaaaaaaaaaaaaaaannnnnnnnatcgaagtgcaggccagt

SEQ ID NO: 36 - DNA, 46 bp, synthetic construct
  source 1..46: mol_type = other DNA; note = Nucleic acid of P2-16
nbaaaaaaaaaaaaaaaaaannnnnnnntcgatacagcaggccagt

SEQ ID NO: 37 - DNA, 46 bp, synthetic construct
  source 1..46: mol_type = other DNA; note = Nucleic acid of P2-17
nbaaaaaaaaaaaaaaaaaannnnnnnntaaggagcgcaggccagt

SEQ ID NO: 38 - DNA, 46 bp, synthetic construct
  source 1..46: mol_type = other DNA; note = Nucleic acid of P2-18
nbaaaaaaaaaaaaaaaaaannnnnnnntgtgtgccgcaggccagt

SEQ ID NO: 39 - DNA, 46 bp, synthetic construct
  source 1..46: mol_type = other DNA; note = Nucleic acid of P2-19
nbaaaaaaaaaaaaaaaaaannnnnnnntgtctgaggcaggccagt

SEQ ID NO: 40 - DNA, 46 bp, synthetic construct
  source 1..46: mol_type = other DNA; note = Nucleic acid of P2-20
nbaaaaaaaaaaaaaaaaaannnnnnnncaacacgtgcaggccagt

SEQ ID NO: 41 - DNA, 20 bp, synthetic construct
  source 1..20: mol_type = other DNA; note = Nucleic acid of A20
  modified_base 20: mod_base = OTHER; note = Fam
aaaaaaaaaaaaaaaaaaaa

SEQ ID NO: 42 - DNA, 33 bp, synthetic construct
  source 1..33: mol_type = other DNA; note = Template conversion primer
aagcagtggtatcaacgcagagtacatrgrgrg

SEQ ID NO: 43 - DNA, 21 bp, synthetic construct
  source 1..21: mol_type = other DNA; note = Second strand synthesis primer
aagcagtggtatcaacgcaga

SEQ ID NO: 44 - DNA, 22 bp, synthetic construct
  source 1..22: mol_type = other DNA; note = Second strand amplification forward primer
ctacacgacgctcttccgatct

SEQ ID NO: 45 - DNA, 22 bp, synthetic construct
  source 1..22: mol_type = other DNA; note = Second strand amplification reverse primer
aagcagtggtatcaacgcagag

SEQ ID NO: 46 - DNA, 33 bp, synthetic construct
  source 1..33: mol_type = other DNA; note = Oligonucleotide linker 1
  modified_base 1: mod_base = OTHER; note = PO4
gatcggaagagcacacgtctgaactccagtcac

SEQ ID NO: 47 - DNA, 13 bp, synthetic construct
  source 1..13: mol_type = other DNA; note = Oligonucleotide linker 2
  modified_base 1: mod_base = OTHER; note = PO4
tctagccttctcg

SEQ ID NO: 48 - DNA, 63 bp, synthetic construct
  source 1..63: mol_type = other DNA; note = Illumina sequencing primer 1
aatgatacggcgaccaccgagatctacacnnnnnnnnnnacactctttccctacacgacgctc

SEQ ID NO: 49 - DNA, 55 bp, synthetic construct
  source 1..55: mol_type = other DNA; note = Illumina sequencing primer 2
caagcagaagacggcatacgagatnnnnnnnnnngtgactggagttcagacgtgt

(19) United States
(12) Patent Application Publication
(10) Pub. No.: US 2024/0263221 A1
(43) Pub. Date: Aug. 8, 2024

(54) Method for high-resolution spatial omic detection of tissue sample

(71) Applicant: JILIN UNIVERSITY, Changchun (CN)

(72) Inventors: Junhu ZHANG, Changchun (CN); Chongyang LIANG, Changchun (CN); Nianzuo YU, Changchun (CN); Zhengyang JIN, Changchun (CN); Bai YANG, Changchun (CN)

(21) Appl. No.: 18/431,976

(22) Filed: Feb. 3, 2024

Related U.S. Application Data
(63) Continuation of application No. PCT/CN2022/110533, filed on Aug. 5, 2022.

(30) Foreign Application Priority Data
Aug. 6, 2021 (CN) 202110899807.3

(51) Int. Cl. C12Q 1/6841; C12Q 1/686

(52) U.S. Cl. CPC C12Q 1/6841; C12Q 1/686

(57) ABSTRACT

A device, system, and method for high-resolution spatial omic detection of a tissue sample are provided, which include a slide with a microwell reaction chamber array capable of accommodating microcarriers, a method for modifying a nucleic acid molecular identifier, and a method for reducing cross pollution of omic information in a process of capturing spatial omic information of a tissue sample, respectively. By using the method for spatial omic detection, the resolution of the spatial omic detection is significantly improved and the detection cost is reduced, and the cross pollution of the spatial omic information is fundamentally reduced.

### CROSS REFERENCE TO RELATED APPLICATION

This patent application is a national stage application of International Patent Application No. PCT/CN2022/110533, filed on Aug. 5, 2022, which claims the benefit and priority of Chinese Patent Application No. 2021108998073 filed with the China National Intellectual Property Administration on Aug. 6, 2021, the disclosure of which is incorporated by reference herein in its entirety as part of the present application.

### REFERENCE TO SEQUENCE LISTING

A computer readable XML file entitled “GWPCTP20240100711”, created on Apr. 18, 2024, with a file size of about 66,143 bytes, contains the sequence listing for this application, has been filed with this application, and is hereby incorporated by reference in its entirety.

### TECHNICAL FIELD

The present disclosure mainly relates to a device, system, and method for high-resolution spatial omic detection of a tissue sample, and in particular, to a method for high-resolution spatial omic detection of a tissue sample.

### BACKGROUND

Human tissues are highly complicated systems made up of many trillions. They may differ in class, time, and space. For example, the tissues of different areas of a mammalian brain have different functions and cell types. In view of this, detection on the spatial heterogeneity of a tissue is particularly important. Spatial omics refers to an omic study that is accomplished on a tissue slice with spatial information of a sample being retained. The spatial omics may show gene expressions in different areas of the tissue slice, reveal the activated signal pathway in a fine pathological area, and resolve the mechanism of a molecular feature driving a pathological feature. The spatial omics accomplishes the technical innovation of pathological digitization combined with pathological imaging and plays an important role in emerging fields such as diagnostic markers, drug resistance sites, and research and development of targeted drugs, and immunological therapy.

Spatial omic detection methods mainly come into four types: a spatial reconstruction method with a computing strategy combined with an omic experiment, a direct measurement method based on laser microdissection, an in situ omics method based on a fluorescent probe and image processing, and an in situ capture technique based on spatial bar coding of oligonucleotides.

The spatial reconstruction method may obtain an intrinsic gene expression trend of cells and an association between the cells by integrating single-cell transcriptomic data in a tissue, but can only present a spatial trend or an overall layout of a particular tissue. The spatial omics technology based on laser capture microdissection (LCM) may realize omic sequencing at single-cell resolution. However, the technology is low in detection flux and is suitable for spatial omic detection of a local tissue. The in situ omics method based on a fluorescent probe and image processing includes two ways: in situ sequencing (ISS) and in situ hybridization (ISH). These methods perform well in detection resolution and may realize spatial omic testing at a subcellular resolution level. However, such methods have high requirements on detection techniques and may need the aid of a high-sensitivity single molecule fluorescence imaging system, and detection needs to undergo complex single molecule hybridization and image analysis processes, which significantly increase the cost and time of spatial omic testing. Therefore, such methods are used only in laboratories at present.

The in situ capture technique based on spatial bar coding of oligonucleotides mainly includes a microsphere assembly technique using fluorescence decoding and the inkjet printing method of 10×Genomics company. The former permits dense stacking of microspheres modified with random coded sequence on a slide surface and utilizes the fluorescence decoding technique to realize decoding and localization of a microsphere sequence. Although such a way increases the spatial coding resolution of a capture sequence, it still relies on the complex and expensive single molecule fluorescence imaging system, and the method is low in oligonucleotide capture efficiency and can hardly realize spatial omic detection of a high-density sequence. At present, the most extensively used spatial omic testing method is the Visium product of the 10×Genomics company. The product uses the inkjet printing method to prepare a high-flux oligonucleotide capture sequence on the surface of a slide. After a tissue sample is attached to the surface of the slide, the tissue is permeabilized. In this process, the sequence to be tested of the tissue sample is captured in situ by the oligonucleotide sequence. By inverse transcription and Next-generation sequencing (NGS), the spatial omic sequencing of the tissue sample is realized. Compared with the several foregoing methods, the method has the advantages of low sequencing cost, high time efficiency, easy operation, and the like. Currently, the 10×Visium product based on this method has been successfully commoditized, and its application range have covered many important research fields.

However, 10×Visium still has several important problems urgently needing to be solved: first, an oligonucleotide sequence array on the surface of a slide is prepared by using the inkjet printing technique. At present, the maximum resolution of sizes of arrays prepared on slides by using this technique is 55 μm, with each sequence dot matrix corresponding to the spatial omic information of a dozen to dozens of cells, which limits single-cell spatial omic analysis and exploration on intercellular interaction mechanism in a tissue sample, and may also result in missing of important information. Second, uneven printing may occur when the inkjet printing method is used to prepare an oligonucleotide sequence array, resulting in a difference in modification effect between the regions of the oligonucleotide sequence and even leading to missed printing, i.e., no sample being printed in a target area. Third, during sequence capture by using the product, a permeabilizing liquid causes a sequence in a tissue sample to diffuse sideways while prompting the sequence to effuse, resulting in cross contamination of the spatial sequence information of the tissue. This phenomenon is particularly serious especially in a high-resolution spatial omic sequencing process. Fourth, this way requires full-length synthesis of oligonucleotide capture sequences at each position in the array, and the synthesis of a large batch of oligonucleotides increases the cost and the design complexity level of the product.

Thus, the defects and limitations of the traditional techniques urgently need to be optimized and solved, which is also the common pursuit and widespread agreement in the industry. Therefore, it is particularly important to develop a method for spatial omic detection of a tissue sample that is high in resolution, low in cost, less in cross contamination, and easy to operate.

### SUMMARY

An objective of embodiments of the present disclosure is to provide a method for high-resolution spatial omic detection of a tissue sample, intended to develop a method for spatial omic detection of a tissue sample that is high in resolution, low in cost, less in cross contamination, and easy to operate.

In a first aspect, the present disclosure provides a slide with a microwell reaction chamber array capable of accommodating microcarriers.

Specifically, a slide with a microwell array is built. Microcarriers are dispersed in the microwell array. Each microwell and the microcarrier therein form a microwell reaction chamber. A molecular identifier is transferred to a microwell reaction chamber. The molecular identifier is ligated to a surface of the microcarrier or the microwell. This kit is used for conducting a spatial omic study on a tissue.

Alternatively, a material of the slide in the skit includes any material usable for preparing a morphological structure, such as glass, silicon, silicon dioxide, and a polymer.

Alternatively, a preparation method for the microwell reaction chambers include any method capable of building a morphological structure. For example, a sunken morphology is etched within the slide from the bottom up, grown on a surface of the slide from the bottom up, or formed by covering the surface of the slide with a substrate with a microwell array or a porous membrane.

Alternatively, a shape of the microwell includes regular and irregular three-dimensional morphological structures, such as cylindrical, circular truncated cone-shaped, and square column-shaped morphological structures.

Alternatively, an internal volume of the microwell reaction chamber ranges from 0.1 fm³ to 1 cm³. Preferably, a volume of the microwell reaction chamber is 10 μm³.

Alternatively, an arrangement of the microwell reaction chamber array includes regular and irregular arrangements. Preferably, the microwell reaction chamber array is arranged as a square array.

Alternatively, each microwell in the microwell reaction chamber array includes at least one microcarrier therein. Alternatively, the microwell may include at least 2, at least 5, at least 10, or at least 100 microcarriers.

Alternatively, the microwell reaction chamber array includes at least one microwell. For example, the microwell array may include at least 10, at least 100, at least 1000, or at least 10000 microwells.

Alternatively, the slide includes at least one microwell reaction chamber array. For example, the slide may include at least 2, at least 10, at least 1000, or at least 1000 microwell reaction chamber arrays.

Alternatively, the microcarriers are microbeads, gels, or polymers capable of being ligated to molecular identifiers, and also include any solid-phase and liquid-phase carriers capable of being ligated to molecular identifiers and any materials capable of generating microcarriers as known to those skilled in the art.

Alternatively, a ligation site at which a molecular identifier is ligated to a microcarrier includes an inside and a surface of the microcarrier, and any other site capable of ligation with the molecular identifier.

Alternatively, an approach of transferring molecular identifiers to the microwell reaction chamber array includes a direct or indirect adding approach. For example, the molecular identifier is transferred to the microwell reaction chamber by inkjet printing or contact printing.

Alternatively, classes of the molecular identifiers include nucleic acid sequence, protein molecule, and other biological molecules. Analyzing and detecting nucleic acid molecular identifiers as described in the present disclosure is also suitable for protein and polysaccharide molecular identifiers, namely including capturing, analyzing, and detecting protein and polysaccharide molecules using the method of the present disclosure.

Alternatively, the molecular identifiers transferred to the microwell reaction chambers may be molecular identifiers different from one another.

Alternatively, the method of the present disclosure includes dispersing the microcarriers that have been ligated with the molecular identifiers in the microwell array. The method of the present disclosure includes transferring the microcarrier that has been ligated with a molecular identifier to the microwell reaction chamber in any way. The method of the present disclosure includes ligating the microcarriers in the microwells with molecular identifiers different from one another in any way and also includes ligating the molecular identifiers within or with surfaces of the microwell reaction chambers.

In a second aspect, the present disclosure provides a method of ligating a unique molecular identifier to a microcarrier.

Specifically, by means of a microchip transfer technique, microchannels arranged in parallel are aligned to the microwell reaction chamber array, and different first molecular identifiers are introduced into the microchannels, respectively. After the first molecular identifiers are ligated with the microcarriers, identifiers not ligated are washed off. The microchannels arranged in parallel are realigned to the microwell reaction chamber array in a direction different from a direction of the microchannel, and different second molecular identifiers are introduced into the microchannels, respectively. The first molecular identifier is conjugated with the second molecular identifier, and the microcarrier is ligated with the unique molecular identifier by extension, amplification, or ligation. The ligating of the unique molecular identifier with the microcarrier includes ligating to the microcarrier described in the present disclosure by any suitable approach.

The first molecular identifier may be a nucleic acid sequence, namely a first nucleic acid molecular identifier, and the sequence includes in a direction from 5′ to 3′: a universal domain, a first location domain, and a ligation domain. The second molecular identifier may be a nucleic acid sequence, namely a second nucleic acid molecular identifier, including in a direction from 3′ to 5′: a ligation domain complementary region, a second location domain, a molecular marker, and a capture domain precursor. The unique molecular identifier may be a nucleic acid sequence, namely a unique nucleic acid molecular identifier, including in the direction from 5′ to 3′: a universal domain, a first location domain, a ligation domain, a second location domain, a molecular marker, and a capture domain. The unique nucleic acid molecular identifier includes a nucleic acid sequence formed after the complementation of the first nucleic acid molecular identifier and the second nucleic acid molecular identifier, and also includes a nucleic acid sequence obtained by extending, amplifying, or ligating the complemented nucleic acid sequence. The “unique” refers to a nucleic acid sequence that is different from other nucleic acid molecular identifiers ligated to the microcarriers and related to cells or tissues and a nucleic acid sequence that is related to the present disclosure.

Alternatively, the classes of the molecular identifiers include nucleic acid sequence, protein molecule, and other biological molecules.

Alternatively, a number of channels of the microchannels arranged in parallel is one or more. For example, parallel microchannels may include at least 10, at least 100, at least 1000, or at least 10000 microchannels arranged in parallel.

Alternatively, a width of a channel of the microchannels arranged in parallel ranges from 0.1 nm to 1000 sm. For example, the parallel microchannels may be each 1, 10, 100, or 1000 μm wide.

Alternatively, a spacing width between channels of the microchannels arranged in parallel ranges from 0.1 nm to 1000 μm. For example, the spacing width between channels may be 1, 10, 100, or 1000 μm.

Alternatively, a liquid inlet arrangement in the microchannels arranged in parallel includes each channel being connected to an independent liquid inlet, also includes a plurality of channels being connected to a same liquid inlet, and also includes one channel being connected to a plurality of liquid inlets.

Alternatively, a liquid outlet arrangement in the microchannels arranged in parallel includes each channel being connected to an independent liquid outlet, and also includes a plurality of channels being connected to a same liquid outlet or one channel being connected to a plurality of liquid outlets.

Alternatively, an approach of ligation between the molecular identifier and the microcarrier includes ligating to the microcarrier by chemical fixation, and also includes a way of promoting the completion of a ligation reaction by a thermal reaction or excitation light of a particular wavelength.

Alternatively, the approach of ligation of the microcarrier and the molecular identifier includes an approach of functionalizing the microcarrier in advance, where the functionalizing is implemented by a chemical, physical, or biological approach, e.g., carrying out the ligation by activating a chemical group in the microcarrier or incorporating an active functional group into the structure of the microcarrier.

Alternatively, the functional group that functionalizes the microcarrier in advance includes a reactive precursor or a precursor that can be activated to form a reactive functional group, e.g., functional groups such as a carboxylic acid group, an aldehyde group, an epoxy group, and 1,4-phenylene diisothiocyanate (PDITC), activated by 1-(3-dimethylaminopropyl)-3-ethylcarbodiimide (EDC) and N-hydroxysuccinimide (NHS).

Alternatively, the universal domain may include a functional group modification site and a universal polymerase chain reaction (PCR) amplification starting end. The functional group modification site includes any reactive substance capable of binding to a microcarrier, and also includes a precursor that can be activated to form a reactive functional group. Any suitable sequence may be used as the universal PCR amplification starting end in the present disclosure. The “suitable sequence” means that the sequence will not influence an interaction between the nucleic acid (e.g., RNA) of a tissue sample and the capture domain, and meanwhile, the sequence may complementarily bind to a universal primer for nucleic acid molecule amplification (e.g., cDNA).

Alternatively, a length of the universal PCR amplification starting end is at least one nucleotide. For example, the length may be 2, 10, 50, 100, or 1000 nucleotides.

Alternatively, the universal domain may include a splicing domain used for releasing a generated nucleic acid molecular identifier from the microcarrier, e.g., poly-U oligonucleotide sequence.

Alternatively, the ligation function of the molecular identifier with the microcarrier may be accomplished by an intrinsic chemical group, or may be accomplished by introducing a group to the molecular identifier.

Alternatively, ways of ligation of the molecular identifier with the microcarrier include but are not limited to physical modification, chemical modification, and biological modification, such as electrostatic binding, amino modification, biotin group modification, phosphorylation modification, photocatalysis modification, and free radical polymerization.

Alternatively, after the nucleic acid molecular identifiers are ligated with the microcarriers, a cleaning step may be carried out. The purpose of the step is to reduce nonspecific adsorption of the nucleic acid molecular identifiers and the microcarriers. This step may be carried out using any known approach in the art. Preferably, a buffer containing components such as a surfactant and a salt may be used.

In the method of the present disclosure, the first location domain may also be defined as a first feature identification domain or a first tag domain, may be regarded as a tag, an identification, or a name of the nucleic acid, and is usually located downstream of and adjacent to the universal domain. The first location domains may be distinguished from one another, and may provide spatial position information for the nucleic acid sequence of the tissue or cell captured by the capture domain (e.g., determining the spatial position of the captured nucleic acid in the tissue or that the captured nucleic acid is derived from a certain cell) without interference with the capture of the nucleic acid sequence of the tissue or cell sample. Any suitable sequence may be used as the first location domain in the present disclosure. The “suitable sequence” means that the sequence will not influence an interaction between the nucleic acid (e.g., RNA) of a tissue sample and the capture domain of a capture probe.

Alternatively, “different” in the different first nucleic acid molecular identifiers refers to being different relative to other molecular identifiers ligated to microcarriers in the present disclosure and other molecular identifiers in the present disclosure, and different first molecular identifiers may provide spatial position information for omic information captured in a tissue or cell (e.g., determining the spatial position of the captured nucleic acid in the tissue or that the captured nucleic acid is derived from a certain cell).

Alternatively, the first location domains of the first molecular identifiers introduced into each of the microchannels arranged in parallel may be different from one another and may be distinguished from one another.

Alternatively, a length of the first location domain sequence is at least one nucleotide. For example, the length may be 2, 10, 50, 100, or 1000 nucleotides.

In the method of the present disclosure, the second location domain may also be defined as a second feature identification domain or a second tag domain, may be regarded as a tag, an identification, or a name of the nucleic acid, and is usually located downstream of and adjacent to the universal domain. The second location domains may be distinguished from one another, and may provide spatial position information for the nucleic acid sequence of the tissue or cell captured by the capture domain (e.g., determining the spatial position of the captured nucleic acid in the tissue or that the captured nucleic acid is derived from a certain cell) without interference with the capture of the nucleic acid sequence of the tissue or cell sample. Any suitable sequence may be used as the second location domain in the present disclosure. The “suitable sequence” means that the sequence will not influence an interaction between the nucleic acid (e.g., RNA) of a tissue sample and the capture domain of a capture probe.

Alternatively, “different” in the different second nucleic acid molecular identifiers refers to being different relative to other molecular identifiers ligated to microcarriers in the present disclosure and other molecular identifiers in the present disclosure, and different second molecular identifiers may provide spatial position information for omic information captured in a tissue or cell (e.g., determining the spatial position of the captured nucleic acid in the tissue or that the captured nucleic acid is derived from a certain cell).

Alternatively, the second location domains of the second molecular identifiers introduced into each of the microchannels arranged in parallel may be different from one another and may be distinguished from one another.

Alternatively, a length of the second location domain sequence is at least one nucleotide. For example, the length may be 2, 10, 50, 100, or 1000 nucleotides.

In the method of the present disclosure, the ligation domain may be any suitable nucleic acid sequence, and the sequence may be complementary to the ligation domain complementary region by Watson-Crick base pairing. The sequence of this part will not influence an interaction between the nucleic acid (e.g., RNA) of a tissue sample and the capture domain of a capture probe and subsequent steps.

In the method of the present disclosure, the ligation domain complementary region may be any suitable nucleic acid sequence, and the sequence may be complementary to the ligation domain by Watson-Crick base pairing. The sequence of this part will not influence an interaction between the nucleic acid (e.g., RNA) of a tissue sample and the capture domain of a capture probe and subsequent steps.

Alternatively, a length of each of sequences of the ligation domain and the ligation domain complementary region is at least one nucleotide. For example, the length may be 2, 10, 50, 100, or 1000 nucleotides.

In the method of the present disclosure, the molecular marker refers to a nucleic acid sequence capable of providing information of a nucleic acid class for hybridization with a nucleic acid molecular identifier. Oligonucleotides conjugated with a same microcarrier may include different molecular markers. The order of the nucleic acid sequence of the molecular marker is unique. The molecular marker may also be defined as a unique molecular identifier (UMI). The molecular marker includes a type for distinguishing between nucleic acids (e.g., mRNAs) hybridized with different nucleic acid molecular identifiers.

Alternatively, a length of the molecular marker is at least one nucleotide. For example, the length may be 2, 10, 50, 100, or 1000 nucleotides.

In the method of the present disclosure, the capture domain precursor may include a nucleic acid sequence for forming a capture domain. The capture domain precursor may include a nucleic acid sequence made up of a poly-A sequence.

Alternatively, a length of the capture domain precursor is at least one nucleotide. For example, the length may be 2, 10, 50, 100, or 1000 nucleotides.

The method of the present disclosure includes the step of co-incubating the hybridized and complemented first and second nucleic acid molecular identifiers and the reaction mixture liquid such that the microcarrier generates the unique nucleic acid molecular identifier (FIG. 4), where the unique nucleic acid molecular identifier includes in the direction from 5′ to 3′: the universal domain, the first location domain, the ligation domain, the second location domain, the molecular marker, and the capture domain, or includes: the universal domain, the first location domain, the second location domain, the molecular marker, and the capture domain. The reaction mixture liquid may include any component capable of extending, amplifying, or ligating a nucleic acid sequence, e.g., components such as a nucleotide, an amplifying enzyme (e.g., DNA polymerase) or a ligase (e.g., T4 DNA ligase), a buffer, and ultrapure water.

Alternatively, the capture domain includes a nucleic acid sequence capable of capturing a nucleic acid sequence.

Alternatively, the capture domain may include a random sequence that may be used in combination with a poly-T oligonucleotide sequence (or poly-T analogue, etc.) to promote the capture of mRNA.

Alternatively, the capture domain may include a completely random sequence, and may also be a degenerate capturing structural domain according to a known principle in the art.

Alternatively, the capture domain may include a poly-T oligonucleotide sequence that may bind to a sequence complementary thereto, e.g., a nucleic acid (e.g., mRNA) carrying a poly-A sequence. The capture domain is not limited to the poly-T oligonucleotide sequence and includes a sequence functionally or structurally similar to the poly-T oligonucleotide sequence, e.g., a poly-U oligonucleotide or a combined oligonucleotide of deoxythymidine analogues, where the oligonucleotide maintains the functional characteristic of binding to a poly-A sequence.

Alternatively, a sequence length of the capture domain may be at least one nucleotide, preferably, at least 5, 10, 15, 20, or 30 nucleotides.

Alternatively, the capture domain may include a nucleic acid sequence capable of guiding a reverse transcription reaction, and may also include a nucleic acid sequence capable of generating a nucleic acid molecule complementary to a captured nucleic acid molecule.

Alternatively, in the method of the present disclosure, an arrangement of functional regions of the first nucleic acid molecular identifier, the second nucleic acid molecular identifier, and the unique nucleic acid molecular identifier includes but is not limited to an order, positions, or contents listed in the present disclosure; and one or more functional sequences of the molecular identifiers may be arranged in any suitable order or contents.

Alternatively, before the reaction mixture liquid is introduced, a prehybridization step may be included, causing the first nucleic acid molecular identifier and the second nucleic acid molecular identifier to be complementary by Watson-Crick base pairing by means of a ligation domain-ligation domain complementary region. This step is beneficial to the generation of a unique nucleic acid molecular identifier.

Preferably, after the generation of the unique nucleic acid molecular identifier, a cleaning step may be carried out, which may clean other substance than the microcarrier and the unique nucleic acid molecular identifier ligated to the microcarrier in the microwell reaction chamber.

Alternatively, the unique nucleic acid molecular identifier may be characterized by any known approach in the art, e.g., using a fluorescently-labeled tag sequence or sequencing analysis, etc.

In a third aspect, the present disclosure provides a method for reducing cross pollution of omic information in a process of capturing spatial omic information of a tissue sample.

Specifically, a solid-phase or liquid-phase compound is introduced into a microwell reaction chamber array in which microcarriers are stored: a tissue slice is attached on a surface of a microwell array; a tissue sample is embedded into microwells or spread on a surface of the microwells, and at this time, the position information of the microcarrier with a specific unique nucleic acid molecular identifier is in one-to-one correspondence with a position of the tissue: the tissue sample is imaged; and the surface of the microwells is covered with a porous membrane for preventing cross pollution between the spatial omic information of the tissue sample. A tissue permeabilizing liquid is added to a surface of the porous membrane, and at this time, the microcarrier captures the nucleic acid sequence of the tissue confined in the microwell by means of the unique nucleic acid molecular identifier, and the surface is cleaned: a reaction mixture liquid is incubated in the microwell array, and a hybrid strand of which the omic information has been captured is extended and synthesized such that the captured nucleic acid sequence and the unique nucleic acid molecular identifier form a complementary double-stranded nucleic acid sequence, followed by amplifying and creating a library for the double-stranded nucleic acid sequence: the nucleic acid sequence is recovered and the recovered nucleic acid sequence is analyzed; next, based on the information of the first location domain and the second location domain, the analyzed tissue or cell sample-derived omic information is caused to correspond to spatial points of the tissue sample by position information, thereby obtaining the spatial omic information of the tissue sample.

The method of the present disclosure may be applied to conduct a spatial transcriptomic study on a tissue.

Specifically, a solid-phase or liquid-phase compound is introduced into the microwell reaction chamber array in which the microcarriers are stored; a tissue slice is attached to a surface of the microwell array; a tissue sample is embedded into the microwells; the surface of the microwells is covered with a porous membrane for preventing cross pollution between the spatial omic information of the tissue sample; a tissue permeabilizing liquid is added to the surface of the porous membrane, and at this time, the microcarrier captures the mRNA of the tissue confined in the microwell by means of the capture domain of the unique nucleic acid molecular identifier, and the surface is cleaned; a reverse transcription reaction mixture liquid is incubated in the microwell array, and a hybrid strand of which the omic information has been captured is extended and synthesized such that the captured mRNA and the unique nucleic acid molecular identifier form cDNA, followed by amplifying and creating a library for cDNA: the nucleic acid sequence is recovered and the recovered nucleic acid sequence is analyzed; next, based on the information of the first location domain and the second location domain, the analyzed tissue or cell sample-derived transcriptomic information is caused to correspond to spatial points of the tissue sample by position information, thereby obtaining spatial transcription information of the tissue sample.

Alternatively, a way of embedding the tissue sample into the microwells includes introducing the tissue sample into the microwells by using any external force or by means of intrinsic properties of the microwell reaction chamber, e.g., by using a mechanical force, or a physical or chemical induction approach.

Alternatively, a class of the solid-phase or liquid-phase compound includes any compound capable of introducing a tissue slice into the microwells, including a polymer, a monomer, and a mixture, such as polyacrylamide, polyvinyl alcohol, polyethylene glycol, dry ice, water, and paraffin.

Alternatively, a volume of the solid-phase or liquid-phase compound added may be greater than, less than, or equal to a volume of the microwell, preferably, equal to the volume of the microwell.

Alternatively, a class of the porous membrane includes a porous membrane of any material, such as a polydimethylsiloxane (PDMS) porous membrane and a polyethylene porous membrane.

Alternatively, a pore diameter of the porous membrane ranges from 0.1 nm to 100 mm, preferably, the pore diameter should be sufficient to cause the tissue permeabilizing liquid to infiltrate while confining a nucleic acid sequence (e.g., mRNA) of a tissue in the microwell within the microwell.

Alternatively, to facilitate analysis of a spatial position of a unique molecular identifier relative to a tissue sample, the tissue sample may be imaged using any known approach in the art, such as light, dark field, and confocal imaging. This step may take place before or after the treatment step for a tissue sample, e.g., before or after a cDNA generation step of the present method.

Alternatively, the tissue sample may be labeled using any known approach in the art so that it can be detected during imaging. For example, tissue staining, fluorescence labeling, or the like may be used.

Alternatively, the permeabilizing liquid may include any liquid, e.g., an enzyme, that causes release of nucleic acid and protein molecules in a cell or tissue sample.

Alternatively, an application range of a method of reducing transverse diffusion in the present disclosure includes capturing any nucleic acid, protein, and polysaccharide molecules in a tissue sample, preferably mRNA molecule, and also includes applying the method to any nucleic acid, protein, and polysaccharide molecules in a cell, such as tRNA, rRNA, and virus RNA.

Alternatively, the method of the present disclosure includes a step of recovering, after a tissue permeabilization step, the microcarriers from the microwell reaction chamber array for a subsequent experiment.

The method of the present disclosure includes an approach of extending the unique nucleic acid molecular identifier and the hybrid strand of the captured nucleic acid to form a complementary double-stranded nucleic acid sequence by any known approach in the art. For example, it is by a reverse transcription reaction. The double-stranded nucleic acid sequence generated by the approach may be regarded as a copy of the captured component of the tissue sample, reflecting the information included in the tissue sample, e.g., transcriptomic information.

Alternatively, the reaction mixture liquid may include any component capable of causing amplification, extension, or ligation of the captured nucleic acid sequence (e.g., mRNA), e.g., a reverse transcription reaction mixture liquid causing reverse transcription of an oligonucleotide sequence into a double-stranded nucleic acid sequence (e.g., cDNA).

Alternatively, the capture domain includes a sequence capable of inducing a captured nucleic acid to generate a complementary strand thereof, and the complementary strand of the captured nucleic acid includes but is not limited to being generated downstream of a unique nucleic acid molecular identifier.

Alternatively, the method of the present disclosure includes a step of removing a nucleic acid strand where the captured nucleic acid is present, e.g., mRNA, after generating the complementary double-stranded nucleic acid sequence. This step may be used to remove the captured nucleic acid by using any known approach in the art, such as a chemical approach, a physical approach, and a biological approach.

Alternatively, in the method of the present disclosure, the tissue slice may be removed after the complementary double-stranded nucleic acid sequence is generated. This step may be carried out using any known approach in the art, e.g., an enzyme degradation approach.

Alternatively, in the method of the present disclosure, the unique molecular identifier may also be amplified before the complementary double-stranded nucleic acid sequence is synthesized.

The method of the present disclosure includes a step of unwinding the complementary double-stranded nucleic acid sequence into an oligonucleotide sequence and synthesizing a complementary strand of the sequence. This step may be understood as generating a second strand of the complementary double-stranded nucleic acid sequence with the purpose of generating the sequence information of the captured nucleic acid of the unique nucleic acid molecular identifier.

Alternatively, in a reaction of generating the second strand, a random primer may be used, whereby a nucleic acid fragment with a random length will be generated, and the nucleic acid product may correspond to the information of the captured sequence.

Alternatively, in the reaction of generating the second strand, a particular primer, e.g., a template conversion primer, may be used, whereby a full-length fragment corresponding to the unique nucleic acid molecular identifier will be generated.

Alternatively, in the reaction of generating the second strand, a template conversion approach may be used. For example, the known SMART technology in the art is adopted. Alternatively, this step may be carried out in situ on the microcarriers in the present disclosure.

Alternatively, in the reaction of generating the second strand, a class of a polymerase used includes any enzyme related to a nucleic acid, such as a DNA polymerase, an RNA polymerase, a DNA ligase, a restriction endonuclease, a transcriptase, and a reverse transcriptase.

Alternatively, in the reaction of generating the second strand, nucleic acid sequence amplification linkers may be introduced, and these sequences may include sites for a polymerase chain reaction or for binding to other amplification and extension reaction primers.

Alternatively, the method of the present disclosure includes a step of recovering the unique nucleic acid molecular identifier or the complementary double-stranded nucleic acid sequence generated by the unique nucleic acid molecular identifier and the captured nucleic acid from the microcarrier. This step may be completed by any known approach in the art, e.g., approaches such as enzymatic splicing release or a high temperature or a salt, with the purpose of destroying the interaction between the nucleic acid and the microcarrier.

The method of the present disclosure includes a step of increasing a number of the second strands (e.g., cDNA). This step may take place on the microcarrier, or may take place after recovering the unique nucleic acid molecular identifier with the information of the captured nucleic acid or the complementary double-stranded nucleic acid sequence thereof from the microcarrier. A number of complementary strands of the nucleic acid that can be generated by this step should be able to be used for a subsequent step, e.g., sequencing analysis.

Alternatively, increasing the number of the second strands (e.g., cDNA) may be accomplished using any known approach in the art, e.g., a polymerase chain reaction. Alternatively, a template for the polymerase chain reaction may be a complementary double-stranded nucleic acid sequence containing a unique nucleic acid molecular identifier, and the product of the reaction may also be used as a template for a subsequent reaction.

The method of the present disclosure includes a step of creating a library for a target sequence containing the information of the captured sequence. This step may be carried out using any known approach in the art. Preferably, a nucleic acid library may be created after Illumina primer sequence is introduced into the amplification product of the second strand by the polymerase chain reaction.

Alternatively, before creating the nucleic acid library, the target sequence may be fragmentated. This step may be beneficial to subsequent library creating and sequencing analysis. This step may be carried out using any known approach in the art, such as a chemical approach, a physical approach, and a biological approach.

Alternatively, before creating the nucleic acid library, a suitable treatment step may be carried out for the target sequence, such as end repair and tailing. This step should be conducive to the creation of the nucleic acid library. This step may be carried out using any known approach in the art, e.g., enzyme treatment.

Alternatively, before creating the nucleic acid library, the primer sequence introduced in the generation reaction of the second strand may be excised. This step may be carried out using any known approach in the art, e.g., a digestion approach.

Alternatively, before creating the nucleic acid library, a step of screening for fragment lengths of amplification products of cDNA may be carried out. This step may be carried out using any known approach in the art, e.g., nucleic acid sequence length analysis.

Alternatively, before creating the nucleic acid library, a particular sequence may be introduced in the target sequence, e.g., a sequencing primer binding site sequence. This step may improve the accuracy of a nucleic acid library analysis result.

Alternatively, before creating the nucleic acid library, a suitable DNA molecule purification method may be used to remove potentially introduced disruptors, such as a non-target nucleic acid sequence, a nucleotide, and a salt. This is conducive to the reliability of a analysis result. This step may be carried out using any known approach in the art, e.g., a magnetic bead separation approach.

Alternatively, when amplifying and creating a library for a target sequence nucleic acid with the captured sequence information, amplifying and library creating approaches include any known nucleic acid amplifying and library creating approaches, such as increasing a sequencing or amplification linker, adding an amplification reaction mixture liquid, and creating a library and amplifying the target sequence.

The method of the present disclosure includes an analysis step for the nucleic acid library. Any approach known in the art may be used to analyze the target nucleic acid sequence. Generally, such an approach is a sequence-specific approach. For example, this approach may use a primer directed to an analyzed sequence, and a sequence analysis approach of an amplification reaction type is adopted. Alternatively, the amplification reaction may be a linear or nonlinear reaction, such as a polymerase chain reaction (PCR) and isothermal amplification reaction (e.g., RPA).

Alternatively, the analysis step may include analysis for the unique molecular identifier, whereby spatial location of the analyzed sequence may be obtained.

Alternatively, each of the complementary double-stranded nucleic acid sequence and the second strand thereof may be analyzed. Analysis processes such as first-generation sequencing, next-generation sequencing, and third-generation sequencing. Alternatively, the sequence analysis approach of the present disclosure may be based on any known means in the art, such as Illumina™ technology and pyrophosphoric acid sequencing.

Alternatively, the method of the present disclosure includes a step of recovering from the microcarrier, creating a library for, and analyzing the unique nucleic acid molecular identifier, or a hybrid strand or a complementary double-stranded nucleic acid sequence generated by the unique nucleic acid molecular identifier and a captured nucleic acid, and any nucleic acid sequence obtained through conversion by the method of the present disclosure. The step may be performed on the microcarrier, or may be performed after recovering the unique nucleic acid molecular identifier with the captured nucleic acid information or the complementary double-stranded nucleic acid sequence thereof.

Alternatively, before the sequence analysis, a suitable DNA molecule purification approach may be used to remove disruptors potentially introduced in the sample, as a non-target nucleic acid sequence, a nucleotide, and a salt. This is conducive to improving the creditability of the result. This step may be carried out using any known approach in the art, e.g., a magnetic bead separation approach.

Alternatively, the tissue sample in the method of the present disclosure may be a tissue sample of any living body or a spatial structure of the living body, such as a plant, an animal, and a fungus.

Alternatively, the tissue sample is a tissue sample of any type or class. For example, a tissue sample of a dead body or a living body and a fresh tissue may all be used as the tissue samples in the present disclosure. The tissue sample in the present disclosure also includes any treated or untreated tissue sample, such as fixed, unfixed, frozen, normal-temperature, and paraffined tissue samples. In one embodiment of the present disclosure, a frozen tissue sample is used. The tissue is embedded by an optimal cutting temperature (OCT) compound which is conducive to maintaining the structure of the tissue and also to slicing of the tissue and which is also compatible with a subsequent step.

Alternatively, the method of the present disclosure may be applied to obtain or retrieve exclusive or independent omic information of an individual cell.

Alternatively, the method of the present disclosure may be applied to omic analysis of any cell or any cell type in a sample, e.g., a blood sample. In other words, cells that the method of the present disclosure is applicable to are not only tissue cells, and may also be a single cell (e.g., a cell separated from an unfixed tissue). The single cell includes a cell fixed at a certain position of the tissue, and also includes a single-cell suspension introduced into the microwells.

Alternatively, the method of the present disclosure may be applied to capture and detect omic information in any biological sample, e.g., to capture DNA, mRNA, protein molecules, tRNA, rRNA, and virus RNA in cell, tissue, and virus samples.

Alternatively, the method of the present disclosure may be applied to any class of biological omic testing and analysis, such as transcriptomics, genomics, epigenomics, proteomics, and metabonomics.

Compared with the prior art, advantages of the present disclosure are as follows:

1. The preparation process is simple in the present disclosure. A high-throughput nucleic acid molecular identifier array with a spatial location domain may be prepared by two microchip operations. An instrument cost required by chip preparation is effectively reduced.

2. The preparation method of the present disclosure avoids full-length synthesis of a capture probe, reduces use classes of desired capture probes, and effectively reduces a material cost required by the preparation of the spatial location domain.

3. A unique nucleic acid molecular identifier array prepared in the present disclosure has a high resolution, and a line width of the array ranges from 0.1 nm to 1000 μm. A single cell resolution may be reached.

4. The unique nucleic acid molecular identifier array prepared in the present disclosure is high in modification density such that the completeness of obtaining spatial omic information in a tissue sample is significantly improved.

5. The unique nucleic acid molecular identifier array prepared in the present disclosure is homogeneous in modification region effect, guaranteeing the homogeneity of the obtained spatial omic information of the tissue sample.

6. The present disclosure effectively reduces transverse diffusion of the spatial omic information in the tissue sample, laying a foundation for the capture of high-resolution spatial omic information.

7. The present method may be expanded to genomics, epigenomics, proteomics, and metabonomics, e.g., applied to analysis of mutations or epigenetic inheritance of tissue cells.

### BRIEF DESCRIPTION OF THE DRAWINGS

FIG. 1 is a diagram illustrating a microwell array for dispersing microcarriers in a method for high-resolution spatial omic detection of a tissue sample provided in an embodiment of the present disclosure;

FIGS. 2A-2B are diagram illustrating a microwell reaction chamber array in a method for high-resolution spatial omic detection of a tissue sample provided in an embodiment of the present disclosure;

FIG. 3 is a diagram illustrating microchip channels for modifying microcarriers with nucleic acid molecular identifiers in a method for high-resolution spatial omic detection of a tissue sample provided in an embodiment of the present disclosure;

FIG. 4 is a diagram illustrating preparation of a microcarrier ligated with a unique nucleic acid molecular identifier in a method for high-resolution spatial omic detection of a tissue sample provided in an embodiment of the present disclosure, * representing a complementary sequence;

FIG. 5 is a diagram illustrating quality testing for microbeads ligated with unique nucleic acid molecular identifiers by fluorescently-labeled poly-A probes in a method for high-resolution spatial omic detection of a tissue sample provided in an embodiment of the present disclosure;

FIG. 6 illustrates a global concept of applying a microwell reaction chamber array filled with microcarriers to high-resolution spatial transcriptomic analysis in a method for high-resolution spatial omic detection of a tissue sample provided in an embodiment of the present disclosure;

FIG. 7 is a diagram illustrating concentration determination of a cDNA amplification product in a method for high-resolution spatial omic detection of a tissue sample provided in an embodiment of the present disclosure;

FIGS. 8A-8B are diagram illustrating quality control for a cDNA amplification product in a method for high-resolution spatial omic detection of a tissue sample provided in an embodiment of the present disclosure;

FIG. 9 is a diagram illustrating concentration determination of a nucleic acid library in a method for high-resolution spatial omic detection of a tissue sample provided in an embodiment of the present disclosure;

FIGS. 10A-10B are diagram illustrating quality control for a nucleic acid library in a method for high-resolution spatial omic detection of a tissue sample provided in an embodiment of the present disclosure; and

FIG. 11 is a diagram illustrating analysis of a nucleic acid library in a method for high-resolution spatial omic detection of a tissue sample provided in an embodiment of the present disclosure.

### DETAILED DESCRIPTION OF THE EMBODIMENTS

In order to make the objectives, technical solutions, and advantages of the present disclosure clearer, the present disclosure is further described in detail below with reference to the accompanying drawings and examples.

The specific implementation of the present disclosure is described in detail below with reference to the accompanying drawings and specific embodiments.

### Example 1: Preparation of Microwell Slide

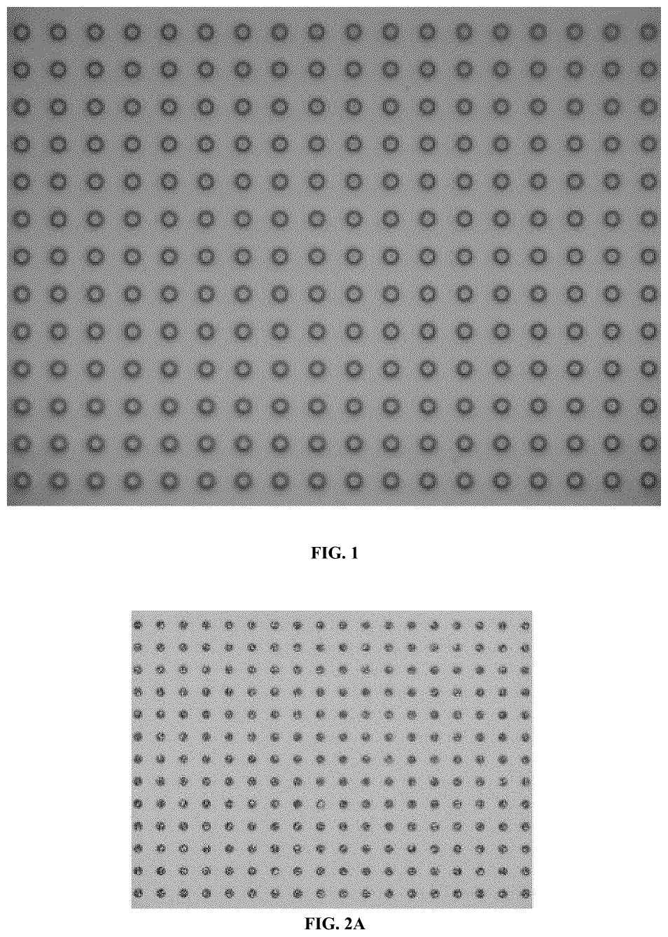

A glass plate with a homogeneous chromium film and a photoresist layer was placed under a mask of a transparent array and exposed to an ultraviolet lamp for 10 s. The substrate was then soaked in a developing solution for 30 s to obtain a patterned photoresist array slide surface spun with a chromium layer. The substrate was then soaked in a chromium etching solution for 5 min. The slide was then soaked in a glass etching solution (in a mass ratio of HF:HNO₃:NH₄F:H₂O=25:23.5:9.35:450) for 80 min to obtain a patterned microwell array slide (FIG. 1), where the microwells were 40 μm high and had a diameter of 30 μm, and a center-to-center spacing of the microwells was 60 μm.

### Example 2: Preparation of Microwell Reaction Chambers

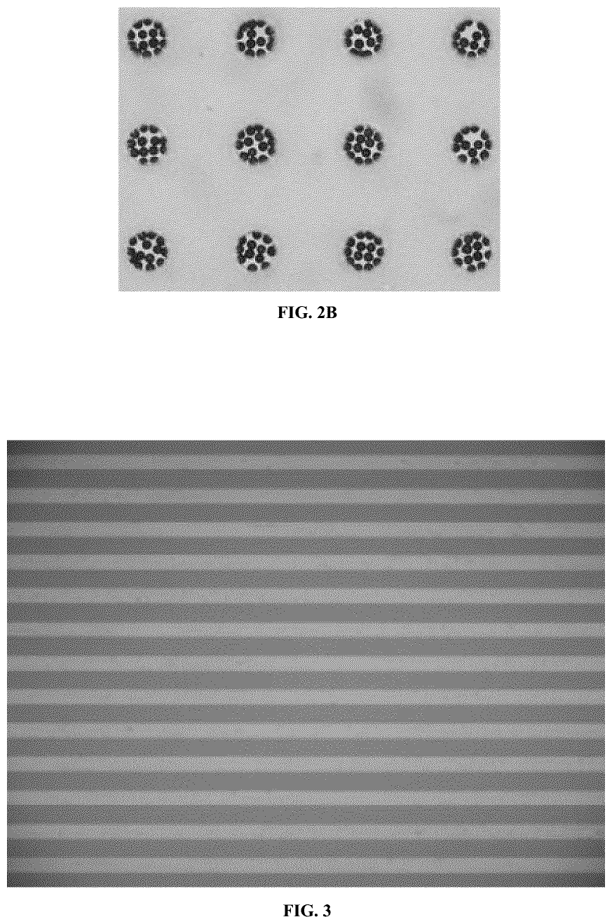

A microbead carrier (having a diameter of 5-7 μm) suspension was spread out on a surface of the microwell slide, and let to stand for 5 min. Microbeads settled down in the microwell array. The surface of the microwell slide was cleaned to wash off the microbeads outside the microwell array to obtain a microwell reaction chamber array containing the microbeads (FIGS. 2A-2B).

### Example 3: Preparation of Microchip

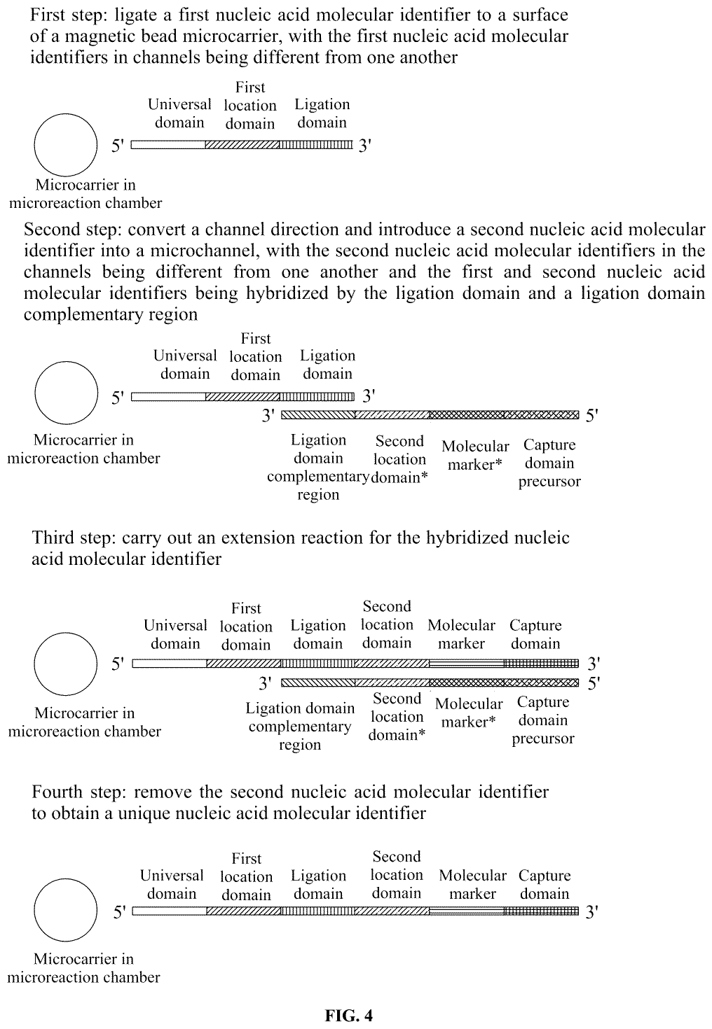

A glass plate with a homogeneous chromium film and a photoresist layer was placed under a mask of channels arranged in parallel and exposed to an ultraviolet lamp for 10 s. The substrate was then soaked in a developing solution for 30 s to obtain a microchannel-patterned photoresist glass surface spun with a chromium layer. The substrate was then soaked in a chromium etching solution for 5 min. The surface was then soaked in a glass etching solution (in a mass ratio of HF:HNO₃:NH₄F:H₂O=25:23.5:9.35:450) for 40 min to obtain a glass channel mold with a microchannel-patterned morphological structure. A polydimethylsiloxane (PDMS) prepolymer and a curing agent were fully mixed in a mass ratio of 10:1, degassed in vacuum for 30 min, then poured onto a surface of the glass mold, and placed in a drying oven at a temperature of 60° C. for curing for 10 h, thereby obtaining PDMS microfluid channels arranged in parallel after being lifted up (FIG. 3). The channels were 20 μm high and 40 μm wide, and a center-to-center spacing of the channels was 60 μm. Apparently, the preparation cost of the microchip was far lower than that of an inkjet printer. Therefore, by using the modification approach with the microchip, the instrument cost of the modification of unique nucleic acid molecular identifiers was reduced significantly.

### Example 4: Preparation and Characterization of Microbeads Ligated with Unique Nucleic Acid Molecular Identifiers

Microbeads having a particle size of 5-7 μm were dispersed in a microwell array including 10000 containable microcarriers, thereby forming 10000 microwell reaction chambers. The microbeads outside the microwell array were removed, and 100 microchannels arranged in parallel were aligned to the microwell reaction chamber array. A first nucleic acid molecular identifier solution was introduced into each channel. The first nucleic acid molecular identifiers of the channels were different from one another (part of sequences shown in Table 1). The microbeads were caused to be ligated with the first nucleic acid molecular identifiers.

TABLE 1
Information of Part of First Nucleic Acid Molecular Identifier Sequences

| Sequence Name | Sequence Information 5′→3′ |
|---|---|
| P1-1 | NH2-CTACACGACGCTCTTCCGATCTGTCACATAACTGGCCTGC |
| | (SEQ ID NO: 1) |
| P1-2 | NH2-CTACACGACGCTCTTCCGATCTCACGCCGGACTGGCCTGC |
| | (SEQ ID NO: 2) |
| P1-3 | NH2-CTACACGACGCTCTTCCGATCTGGTCCAGGACTGGCCTGC |
| | (SEQ ID NO: 3) |
| P1-4 | NH2-CTACACGACGCTCTTCCGATCTATCGAATGACTGGCCTGC |
| | (SEQ ID NO: 4) |
| P1-5 | NH2-CTACACGACGCTCTTCCGATCTCTAGTATAACTGGCCTGC |
| | (SEQ ID NO: 5) |
| P1-6 | NH2-CTACACGACGCTCTTCCGATCTATGGCTAAACTGGCCTGC |
| | (SEQ ID NO: 6) |
| P1-7 | NH2-CTACACGACGCTCTTCCGATCTAGACATGCACTGGCCTGC |
| | (SEQ ID NO: 7) |
| P1-8 | NH2-CTACACGACGCTCTTCCGATCTAGGCGATCACTGGCCTGC |
| | (SEQ ID NO: 8) |
| P1-9 | NH2-CTACACGACGCTCTTCCGATCTTGTCCTTAACTGGCCTGC |
| | (SEQ ID NO: 9) |
| P1-10 | NH2-CTACACGACGCTCTTCCGATCTGATTAGGAACTGGCCTGC |
| | (SEQ ID NO: 10) |
| P1-11 | NH2-CTACACGACGCTCTTCCGATCTTGGATCCAACTGGCCTGC |
| | (SEQ ID NO: 11) |
| P1-12 | NH2-CTACACGACGCTCTTCCGATCTCATAAGCGACTGGCCTGC |
| | (SEQ ID NO: 12) |
| P1-13 | NH2-CTACACGACGCTCTTCCGATCTACTGTATTACTGGCCTGC |
| | (SEQ ID NO: 13) |
| P1-14 | NH2-CTACACGACGCTCTTCCGATCTCCTTGCGGACTGGCCTGC |
| | (SEQ ID NO: 14) |
| P1-15 | NH2-CTACACGACGCTCTTCCGATCTAGGTGTAGACTGGCCTGC |
| | (SEQ ID NO: 15) |
| P1-16 | NH2-CTACACGACGCTCTTCCGATCTGGAGATTAACTGGCCTGC |
| | (SEQ ID NO: 16) |
| P1-17 | NH2-CTACACGACGCTCTTCCGATCTCGATTACAACTGGCCTGC |
| | (SEQ ID NO: 17) |
| P1-18 | NH2-CTACACGACGCTCTTCCGATCTCCGGATAGACTGGCCTGC |
| | (SEQ ID NO: 18) |
| P1-19 | NH2-CTACACGACGCTCTTCCGATCTCACTTGGAACTGGCCTGC |
| | (SEQ ID NO: 19) |
| P1-20 | NH2-CTACACGACGCTCTTCCGATCTGAGAAGTTACTGGCCTGC |
| | (SEQ ID NO: 20) |

A cleaning liquid was introduced into the microchannels to wash off the first nucleic acid molecular identifiers not ligated. The 100 microchannels arranged in parallel were realigned to the microwell reaction chamber array in a direction perpendicular to the above channel direction. Second nucleic acid molecular identifiers were introduced into the microchannels, and the second nucleic acid molecular identifiers of the channels were different from one another (part of sequences shown in Table 2). The ligation domains of the second nucleic acid molecular identifier and the first nucleic acid molecular identifier were hybridized and complemented by a ligation domain complementary region. The cleaning liquid was then introduced to wash off the second nucleic acid molecular identifiers not hybridized. In this embodiment, 200 nucleic acid molecular identifiers could be used to realize modification of 10000 different unique nucleic acid molecular identifier microbead arrays. This obviously reduced the reagent cost required by full-length sequence synthesis in an inkjet printing modification approach in the prior art.

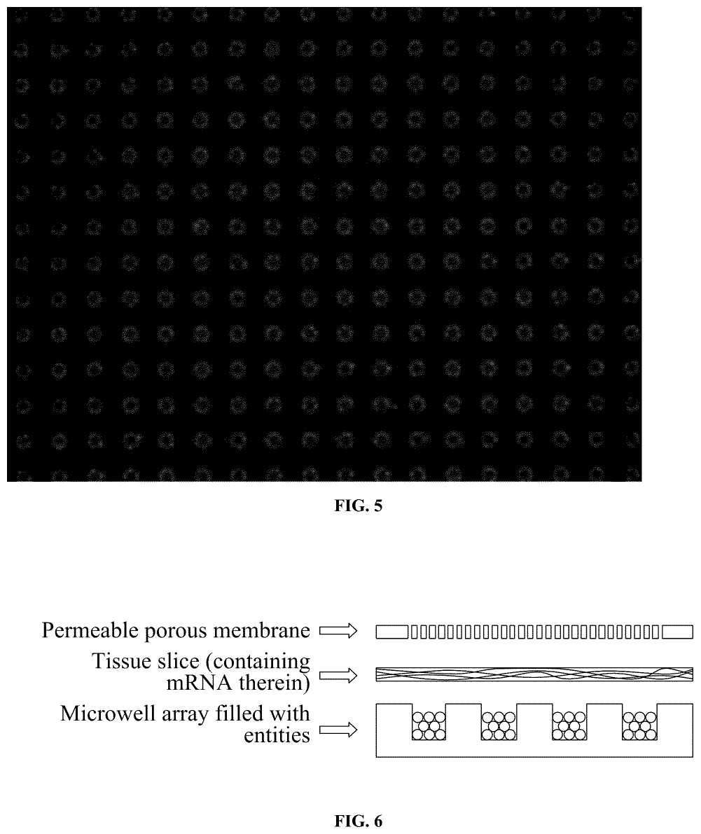

An amplification reaction mixture liquid was introduced into the microchannels for incubation at a constant temperature to generate the unique nucleic acid molecular identifiers, each of which was composed of, from end 5′ to end 3′, a universal domain, a first location domain, a ligation domain, a second location domain, a molecular marker, and a capture domain. The cleaning liquid was introduced into the microchannels to clean the microbeads in the microwell reaction chambers, thereby obtaining the microbeads ligated with the unique nucleic acid molecular identifiers (FIG. 4). A fluorescently-labeled nucleic acid sequence A20 capable of being hybridized and complemented with the unique nucleic acid molecular identifier was added dropwise to the surface of the microwell reaction chamber array (see Table 3). A fluorescence microscope was adopted for characterization, and a modification result showed that the microbeads had been ligated with the unique nucleic acid molecular identifiers (FIG. 5). Amplification sequencing was performed on the unique nucleic acid molecular identifier, and a result also showed that the composition of the unique nucleic acid molecular identifier was correct, which contained the location domain, the molecular marker, and the capture domain. The modification resolution of the unique nucleic acid molecular identifier was 30 μm. In some embodiments, the modification resolution of the unique nucleic acid molecular identifier was obviously higher than that of the existing inkjet printing modification approach. Moreover, in this embodiment, the preparation density of the unique nucleic acid molecular identifiers was high with a spacing of 30 μm, which was lower than that of the modification approach in the prior art. The completeness of obtaining spatial omic information in a tissue sample was significantly improved. In this embodiment, the unique nucleic acid molecular identifier array prepared in the present disclosure was homogeneous in modification effect, guaranteeing the homogeneity of the obtained spatial omic information of the tissue sample.

TABLE 2
Information of Part of Second Nucleic Acid Molecular Identifier Sequences

| Sequence Name | Sequence Information 5′→3′ |
|---|---|
| P2-1 | NBAAAAAAAAAAAAAAAAAANNNNNNNNATATTGTGGCAGGCCAGT |
| | (SEQ ID NO: 21) |
| P2-2 | NBAAAAAAAAAAAAAAAAAANNNNNNNNCTAGGTGTGCAGGCCAGT |
| | (SEQ ID NO: 22) |
| P2-3 | NBAAAAAAAAAAAAAAAAAANNNNNNNNTTGCGTTCGCAGGCCAGT |
| | (SEQ ID NO: 23) |
| P2-4 | NBAAAAAAAAAAAAAAAAAANNNNNNNNGTACGACTGCAGGCCAGT |
| | (SEQ ID NO: 24) |
| P2-5 | NBAAAAAAAAAAAAAAAAAANNNNNNNNCTGTATTTGCAGGCCAGT |
| | (SEQ ID NO: 25) |
| P2-6 | NBAAAAAAAAAAAAAAAAAANNNNNNNNTCTGCGCCGCAGGCCAGT |
| | (SEQ ID NO: 26) |
| P2-7 | NBAAAAAAAAAAAAAAAAAANNNNNNNNCGATCATTGCAGGCCAGT |
| | (SEQ ID NO: 27) |
| P2-8 | NBAAAAAAAAAAAAAAAAAANNNNNNNNTTCTCTTGGCAGGCCAGT |
| | (SEQ ID NO: 28) |
| P2-9 | NBAAAAAAAAAAAAAAAAAANNNNNNNNTAGAGATCGCAGGCCAGT |
| | (SEQ ID NO: 29) |
| P2-10 | NBAAAAAAAAAAAAAAAAAANNNNNNNNCGCGTGTTGCAGGCCAGT |
| | (SEQ ID NO: 30) |
| P2-11 | NBAAAAAAAAAAAAAAAAAANNNNNNNNTAACTACCGCAGGCCAGT |
| | (SEQ ID NO: 31) |
| P2-12 | NBAAAAAAAAAAAAAAAAAANNNNNNNNCCCTCCTGGCAGGCCAGT |
| | (SEQ ID NO: 32) |
| P2-13 | NBAAAAAAAAAAAAAAAAAANNNNNNNNTAAGCGGAGCAGGCCAGT |
| | (SEQ ID NO: 33) |
| P2-14 | NBAAAAAAAAAAAAAAAAAANNNNNNNNTACTAGCAGCAGGCCAGT |
| | (SEQ ID NO: 34) |
| P2-15 | NBAAAAAAAAAAAAAAAAAANNNNNNNNATCGAAGTGCAGGCCAGT |
| | (SEQ ID NO: 35) |
| P2-16 | NBAAAAAAAAAAAAAAAAAANNNNNNNNTCGATACAGCAGGCCAGT |
| | (SEQ ID NO: 36) |
| P2-17 | NBAAAAAAAAAAAAAAAAAANNNNNNNNTAAGGAGCGCAGGCCAGT |
| | (SEQ ID NO: 37) |
| P2-18 | NBAAAAAAAAAAAAAAAAAANNNNNNNNTGTGTGCCGCAGGCCAGT |
| | (SEQ ID NO: 38) |
| P2-19 | NBAAAAAAAAAAAAAAAAAANNNNNNNNTGTCTGAGGCAGGCCAGT |
| | (SEQ ID NO: 39) |
| P2-20 | NBAAAAAAAAAAAAAAAAAANNNNNNNNCAACACGTGCAGGCCAGT |
| | (SEQ ID NO: 40) |

### Example 5: Treatment of Tissue Sample

A container holding isopentane and a collecting instrument were placed in liquid nitrogen to be precooled for 10 min, and then fresh mouse brain tissue was immersed in the isopentane until the tissue was completely frozen, and then transferred to storage at −80° C. Using a precooled instrument, the frozen mouse brain tissue was placed on a precooled OCT compound (a mixture of polyethylene glycol and polyvinyl alcohol), and the exposed surface of the tissue was covered with the OCT compound. After confirming that there was no bubble around the tissue, the tissue was immediately placed on dry ice until the OCT compound was completely frozen. The tissue was cut into a suitable size and immediately sliced under a freezing condition (or transferred to sealed storage at −80° C.).

A freezing microtome was precooled in advance, and a cutting thickness was adjusted to 10 μm. The microwell reaction chamber array was then placed in the freezing microtome to be precooled, and the OCT frozen tissue sample was placed on the microwell reaction chamber array to cut a tissue slice having a thickness of 10 μm.

### Example 6: Staining of Tissue Sample

The microwell reaction chamber array was placed on a preheated PCR amplifier with the side of the slice facing upwards for incubation under the condition of 37° C. for 1 min. The tissue slice was fixed using precooled methanol under the condition of −20° C. for 30 min. the methanol on the surface of the array was then removed, and the slice sample was incubated with isopropanol at room temperature for 1 min and air-dried. A hematoxylin staining liquid was added dropwise to cover the slice sample and incubated for more than 5 min and less than 10 min, and the staining liquid was discarded and the microwell reaction chamber array was thoroughly cleaned. A bluing staining liquid was added dropwise to cover the slice sample and incubated for 2 min, and the staining liquid was discarded and the microwell reaction chamber array was thoroughly cleaned. An eosin staining liquid (eosin:trihydroxymethyl aminomethane buffer (pH=6.0)=1:9) was added dropwise to cover the slice sample and incubated for 1 min, and the staining liquid was discarded and the microwell reaction chamber array was thoroughly cleaned. Finally, the microwell reaction chamber array was incubated at 37° C. for more than 5 min and less than 10 min. A bright field microscope was used to take a photo, and an exposure time and a shooting range were adjusted such that the boundary of the microwell reaction chamber array was visible.

### Example 7: Tissue Permeabilization and cDNA Synthesis

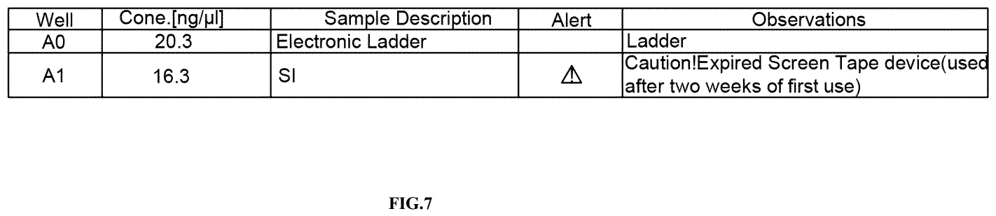

A semipermeable membrane was attached on the microwell reaction chamber array (FIG. 6), and then a tissue permeabilizing liquid (0.1% pepsase, 0.1 M hydrochloric acid) was added dropwise to the semipermeable membrane and incubated at 37° C. for 30 min. The liquid was then removed, and a sodium citrate buffer was added to the microwell reaction chamber array for cleaning, and the semipermeable membrane and the waste liquid were removed. A reverse transcription reaction liquid (containing a hot start reverse transcriptase and a corresponding buffer system and template conversion primer, the template conversion primer being shown in Table 3) was added to the microwell reaction chamber array and incubated at 50° C. for 1 h, and then the reverse transcription reaction liquid was removed.

A KOH solution (100 mM) was added dropwise to the microwell reaction chamber array, incubated at room temperature for 3 min, and then removed, and a trihydroxymethyl aminomethane buffer (10 mM Tris-HCl, pH=8.5) was added. The buffer was removed from the microwell reaction chamber array. A second strand synthesis reaction liquid (containing Bst 2.0 polymerase and a corresponding buffer system as well as a corresponding primer, the primer sequence being shown in Table 3), incubated at 65° C. for 30 min, and then removed, and the trihydroxymethyl aminomethane buffer (10 mM Tris-HCl, pH=8.5) was added. The buffer was removed again, and the KOH solution (100 mM) was added and incubated at room temperature for 5 min. The solution was then transferred to an EP tube without nuclease pollution, and a trihydroxymethyl aminomethane buffer (1 M Tris-HCl, pH=7.2) was added and stored at a low temperature.

### Example 8: cDNA Amplification

A mixture of a second strand synthesis product and a trihydroxymethyl aminomethane buffer was placed on ice, added with a cDNA second strand amplification reaction liquid (Taq enzyme, dNTP, and a corresponding buffer system) and a primer (a primer sequence shown in Table 3), and fully mixed for a polymerase chain reaction, under the following conditions:
Step 1: 98° C., 3 min;
Step 2: 98° C. 15 s;
Step 3: 63° C. 20 s;
Step 4: 72° C., 60 s;
Step 5: skipping to step 2, and a total of 21 cycles;
Step 6: 72° C., 60 s;
Step 7: 4° C., end.

The product was stored at −20° C. for later use.

The primer information was shown in Table 3.

### Example 9: Purification of cDNA Amplification Product

SPRIselect nucleic acid fragment selection kit (Beckman Coulter) was used. The amplification product was mixed with SPRiselect solution, and let to stand at room temperature for more than 5 min. Magnetic beads were separated by a magnet, and the supernatant was discarded. An 80% ethanol solution was added to the magnetic beads, and let to stand for 30 s. The magnetic beads were then separated by the magnet, and the supernatant was discarded. This process was repeated twice and the residual ethanol was removed thoroughly. The trihydroxymethyl aminomethane buffer (10 mM Tris-HCl, pH=8.5) was added, and let to stand for 2 min after being blown and beaten using a pipette. The magnetic beads were separated by the magnet, and the supernatant was retained and stored at a low temperature. An automatic electrophoresis system (Agilent Technologies) was used to determine a size distribution of cDNA fragments, and NanoDrop was used to determine a cDNA concentration.

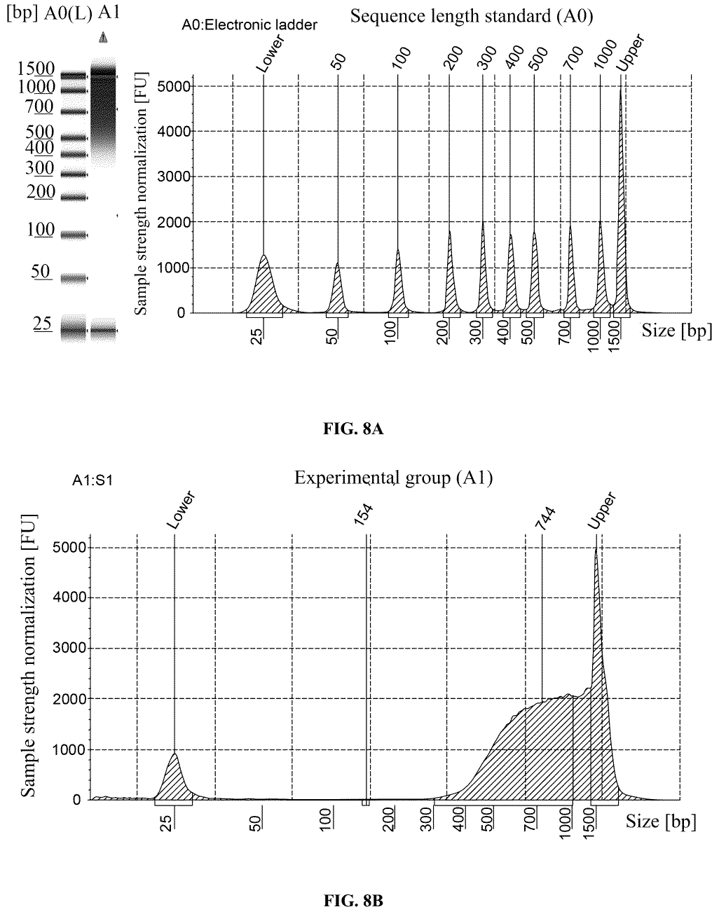

The cDNA amplification product concentration and the purification result were as shown in FIG. 7 and FIGS. 8A-8B. A hybrid sequence in the microwell reaction chamber after capture of mRNA was subjected to reverse transcription, and the resulting cDNA was enriched and collected. Next, the distribution and the concentration of cDNA were determined, and the detection result showed that 300-1000 bp cDNA sequence was generated, which was consistent with the expected result.

### Example 10: Library Creation for cDNA Amplification Product

The cDNA purification product was transferred to ice, added with precooled trihydroxymethyl aminomethane buffer (10 mM Tris-HCl, pH=8.5) and primer excision liquid and fully mixed, and then transferred to a PCR amplifier preset to 4° C. and then incubated under the condition of 32° C. for 5 min. SPRiselect solution was added to the reaction product, fully mixed, and let to stand at room temperature for 5 min. Magnetic beads were separated by a magnet, and the supernatant was transferred to a new EP tube, added with the SPRIselect solution, fully mixed, and let to stand at room temperature for 5 min. The magnetic beads were separated by the magnet, and the supernatant was discarded. An 80% ethanol solution was added to the magnetic beads, and let to stand for 30 s. The magnetic beads were then separated by the magnet, and the supernatant was discarded. This process was repeated twice and the residual ethanol was removed thoroughly. The trihydroxymethyl aminomethane buffer (10 mM Tris-HCl, pH=8.5) was added, and let to stand for 2 min after being blown and beaten using a pipette. Part of the supernatant was taken, added with a cDNA modification reaction liquid (containing a DNA ligase and a corresponding buffer system as well as an oligonucleotide linker, the oligonucleotide linker being shown in Table 3), fully mixed, and incubated under the condition of 20° C. for 20 min. The reaction product was mixed with SPRIselect solution, and let to stand at room temperature for more than 5 min. The magnetic beads were separated by the magnet, and the supernatant was discarded. The 80% ethanol solution was added to the magnetic beads, and let to stand for 30 s. The magnetic beads were then separated by the magnet, and the supernatant was discarded. This process was repeated twice and the residual ethanol was removed thoroughly. The trihydroxymethyl aminomethane buffer (10 mM Tris-HCl, pH=8.5) was added, and let to stand for 2 min after being blown and beaten using the pipette. The magnetic beads were separated by the magnet. Part of the supernatant was suctioned into a new EP tube, added with a PCR reaction liquid (Taq enzyme, dNTP, and a corresponding buffer system) and Illumina primer (Table 3), and fully mixed for a polymerase chain reaction, under the following conditions:
Step 1: 98° C., 1 min;
Step 2: 98° C., 20 s;
Step 3: 63° C., 30 s;
Step 4: 72° C., 20 s;
Step 5: skipping to step 2, and a total of 12 cycles;
Step 6: 72° C., 60 s;
Step 7: 4° C., end.

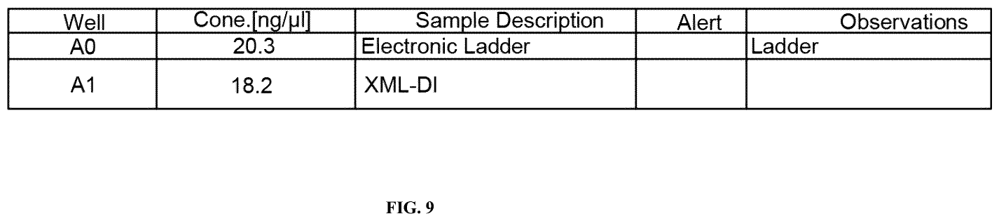

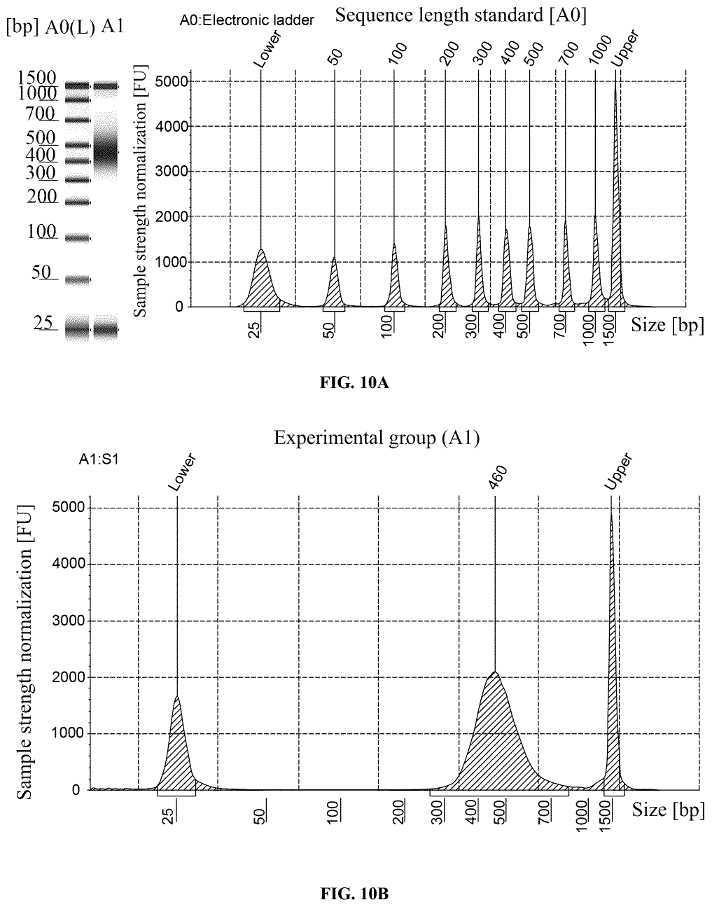

The nucleic acid library product was mixed with SPRIselect solution, and let to stand at room temperature for more than 5 min. The magnetic beads were separated by the magnet, and the supernatant was discarded. The 80% ethanol solution was added to the magnetic beads, and let to stand for 30 s. The magnetic beads were then separated by the magnet, and the supernatant was discarded. This process was repeated twice and the residual ethanol was removed thoroughly. The trihydroxymethyl aminomethane buffer (10 mM Tris-HCl, pH=8.5) was added, and let to stand for 2 min after being blown and beaten using a pipette. The magnetic beads were separated by the magnet, and the supernatant was retained and stored at a low temperature. An automatic electrophoresis system (Agilent Technologies) was used to determine a size distribution of nucleic acid fragments, and NanoDrop was used to determine a nucleic acid concentration. The concentration and the distribution of the amplified nucleic acid library were as shown in FIG. 9 and FIGS. 10A-10B.

The related nucleic acid sequence information was as shown in Table 3, and the concentration and distribution results of the amplified nucleic acid library were as shown in FIG. 9 and FIGS. 10A-10B. A library was created for cDNA derived from the tissue and the unique nucleic acid molecular identifiers in the microwell reaction chambers. The sequence distribution after the creation of the library was determined. The detection result showed that the nucleic acid sequence having a length of 300-700 bp was generated, which was consistent with the expected result.

TABLE 3
Part of Related Sequences in Examples

| Sequence Name | Sequence Information 5′→3′ |
|---|---|
| A₂₀ | AAAAAAAAAAAAAAAAAAAA-Fam (SEQ ID NO: 41) |
| Template conversion | AAGCAGTGGTATCAACGCAGAGTACATrGrGrG (SEQ ID NO: 42) |
| primer | |
| Second strand synthesis | AAGCAGTGGTATCAACGCAGA (SEQ ID NO: 43) |
| primer | |
| Second strand | Forward primer: CTACACGACGCTCTTCCGATCT (SEQ ID NO: 44) |
| amplification primer | Reverse primer: AAGCAGTGGTATCAACGCAGAG (SEQ ID NO: 45) |
| Oligonucleotide linker | 5′PO4-GATCGGAAGAGCACACGTCTGAACTCCAGTCAC (SEQ ID NO: 46) |
| | 5′PO4-TCTAGCCTTCTCG (SEQ ID NO: 47) |
| Illumina sequencing | AATGATACGGCGACCACCGAGATCTACACNNNNNNNNNNACAC |
| primer 1 | TCTTTCCCTACACGACGCTC (SEQ ID NO: 48) |
| Illumina sequencing | CAAGCAGAAGACGGCATACGAGATNNNNNNNNNNGTGACTGG |
| primer 2 | AGTTCAGACGTGT (SEQ ID NO: 49) |

### Example 11: Analysis of Nucleic Acid Library

Novaseq was selected for sequencing for the library. Sequence analysis was performed on the sequenced nucleic acid library to determine the position information of the nucleic acid sequence in combination with the sequence information of the nucleic acid molecular identifiers in the magnetic bead microcarriers. The expression information of a desired gene at a particular position in space was determined by matching between target genes. The expressions of the desired gene were counted and quantified by counting the molecular markers corresponding to the desired gene. This way may reduce data errors caused by the amplification biases of the desired gene. The sequencing data was processed. The unique nucleic acid molecular identifier array of each spatial point was matched with a tissue stained image of the tissue slice such that the sequencing data of the nucleic acid library was visualized at the spatial position of the tissue slice.

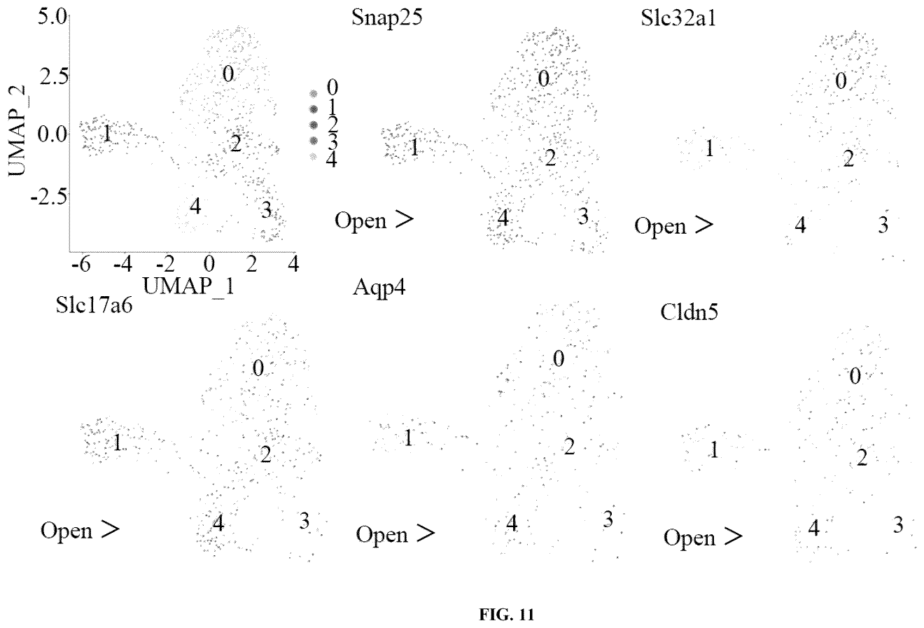

The result showed that all the corresponding nucleic acid molecular identifier information could be found in the analysis result of the nucleic acid library, the sequence information of the first location domain, the second location domain, and the molecular marker was correct, and the data quality could meet the analysis requirement. The data analysis result showed that each microwell reaction chamber could capture more than 2000 genes. The quality of nFeature, nCount, and mito statistical data was good; the umap grouping result was correct; and the typical gene staining results of Snap25, Slc32a1, Slc17a6, Aqp4, and Cldn5 brain tissues were correct (FIG. 11).

The above described are merely preferred embodiments of the present disclosure, and not intended to limit the present disclosure. Any modifications, equivalent replacements and improvements made within the spirit and principle of the present disclosure should all fall within the scope of protection of the present disclosure.

In addition, it should be noted that various specific technical features described in the above specific embodiments can be combined in any suitable manner, provided that there is no contradiction. To avoid unnecessary repetition, various possible combination modes of the present disclosure are not described separately.

In addition, different embodiments of the present disclosure can also be combined arbitrarily. The combinations should also be regarded as the contents disclosed in the present disclosure, provided that they do not violate the ideas of the present disclosure.

1. A method for high-resolution spatial omic detection of a tissue sample, comprising: step a, a device comprising a slide with a microwell array, dispersing microcarriers in the microwell array, with each microwell and a microcarrier in each microwell forming a microwell reaction chamber; and further comprising:
step b, transferring a first molecular identifier to a microwell reaction chamber, and ligating the first molecular identifier to a microcarrier or microwell surface, wherein the first molecular identifier is transferred to the microwell reaction chamber using a microchip transfer technique; specifically, microchannels arranged in parallel are aligned to a microwell reaction chamber array; different first molecular identifiers are introduced into the microchannels, respectively; the first molecular identifiers are ligated to the microcarriers; the first molecular identifier is preferably a nucleic acid sequence, namely a first nucleic acid molecular identifier, and the sequence preferably comprises in a direction from 5′ to 3′:
(i) a universal domain;
(ii) first location domains, wherein the first location domains are different from one another and preferably distinguished from one another, and information of the sequence corresponds to a liquid position where the sequence is introduced into the microchannel; and
(iii) a ligation domain, the sequence being used for ligating first and second nucleic acid molecular identifiers;
step c, transferring a second molecular identifier to the microwell reaction chamber, wherein the second molecular identifier is transferred to the microwell reaction chamber using the microchip transfer technique; specifically, the microchannels arranged in parallel are realigned to the microwell reaction chamber array in a direction different from a direction of the microchannel; different second molecular identifiers are introduced into the microchannels, respectively; the first molecular identifier is conjugated with the second molecular identifier; the second molecular identifier is preferably a nucleic acid sequence, namely a second nucleic acid molecular identifier, preferably comprising in a direction from 5′ to 3′:
a) a ligation domain complementary region, preferably hybridized with the ligation domain by Watson-Crick base pairing;
b) second location domains, wherein the second location domains are different from one another and capable of being distinguished from one another, and information of the sequence corresponds to a liquid position where the sequence is introduced into the microchannel; and
c) a molecular marker, used for providing information of a nucleic acid class for hybridization with a nucleic acid molecular identifier and distinguishing between nucleic acid types for hybridization with different nucleic acid molecular identifiers, wherein the second nucleic acid molecular identifiers conjugated with a same microcarrier preferably comprise different molecular markers; and
d) a capture domain precursor, comprising a nucleic acid sequence for forming a capture domain;
wherein “different” in the different first/second nucleic acid molecular identifiers refers to being different relative to a molecular identifier ligated to a microcarrier and other molecular identifiers, and different first/second molecular identifiers preferably provide spatial position information for omic information captured in a tissue or cell; and
step d, co-incubating the hybridized and complemented first and second molecular identifiers and a reaction mixture liquid, and ligating a microcarrier to a unique molecular identifier by extension, amplification, or ligation, wherein the ligating of the unique molecular identifier with the microcarrier comprises ligating to the microcarrier by any appropriate approach; the unique molecular identifier is preferably a nucleic acid sequence, namely a unique nucleic acid molecular identifier, preferably comprising in the direction from 5′ to 3′:
1) a universal domain;
2) a first location domain;
3) a ligation domain;
4) a second location domain;
5) a molecular marker; and
6) a capture domain, preferably comprising a nucleic acid sequence capable of capturing a nucleic acid sequence, a random sequence, a degenerate capturing structural domain, a sequencing and promoter linker sequence, or a combination thereof, or comprising a sequence functionally or structurally similar to a poly-T oligonucleotide sequence;
wherein the unique nucleic acid molecular identifier comprises a nucleic acid sequence formed after the complementation of the first nucleic acid molecular identifier and the second nucleic acid molecular identifier or a nucleic acid sequence obtained by extending, amplifying, or ligating the complemented nucleic acid sequence; and the unique nucleic acid molecular identifier refers to a nucleic acid sequence that is different from other nucleic acid molecular identifiers ligated to the microcarriers and related to cells or tissues and a nucleic acid sequence that is related to the present disclosure.

2. The method for high-resolution spatial omic detection of a tissue sample according to claim 1, wherein a spatial omic study is conducted on a tissue sample, and cross pollution of omic information is reduced: specifically, a solid-phase or liquid-phase compound is introduced into the microwell reaction chamber array in which the microcarriers are stored; a tissue slice is attached to a surface of the microwell array; the tissue sample is embedded into microwells or spread out on a surface of the microwells, and the position information of the microcarrier with a specific unique nucleic acid molecular identifier is in one-to-one correspondence with a position of the tissue; the tissue sample is imaged; the surface of the microwells is covered with a porous membrane for preventing cross pollution between the spatial omic information of the tissue sample; a tissue permeabilizing liquid is added to a surface of the porous membrane, and the microcarrier captures the nucleic acid sequence of the tissue confined in the microwell, and the surface is cleaned; a reaction mixture liquid is incubated in the microwell array, and a hybrid strand of which the omic information has been captured is extended and synthesized such that the captured nucleic acid sequence and the unique nucleic acid molecular identifier form a complementary double-stranded nucleic acid sequence, followed by amplifying and creating a library for the double-stranded nucleic acid sequence; the nucleic acid sequence is recovered and the recovered nucleic acid sequence is analyzed; next, based on sequence positions and imaging detection information of the first location domain and the second location domain, the analyzed tissue or cell sample-derived omic information is caused to correspond to spatial points of the image of the tissue sample by position information, thereby obtaining the spatial omic information of the tissue sample.

3. The method for high-resolution spatial omic detection of a tissue sample according to claim 1, wherein the microcarriers dispersed in the microwell array comprise a microcarrier that has been ligated with a molecular identifier; the method comprises transferring the microcarrier that has been ligated with a molecular identifier to the microwell reaction chamber in any way; the method comprises ligating the microcarriers in the microwells with molecular identifiers different from one another in any way and also comprises ligating the molecular identifiers within or with surfaces of the microwell reaction chambers.

4. The method for high-resolution spatial omic detection of a tissue sample according to claim 1, wherein classes of the molecular identifiers comprise a nucleic acid sequence, a protein molecule, and a polysaccharide molecule; and analyzing and detecting nucleic acid molecular identifiers by the method is also suitable for protein and polysaccharide molecular identifiers, namely comprising capturing, analyzing, and detecting protein and polysaccharide molecules using the method.

5. The method for high-resolution spatial omic detection of a tissue sample according to claim 1, wherein the microcarriers are microbeads, gels, or polymers ligated to molecular identifiers, and also comprise any solid-phase and liquid-phase carriers capable of being ligated to molecular identifiers and any materials capable of generating microcarriers as known to those skilled in the art; and a ligation site of a molecular identifier and a microcarrier comprises an inside and a surface of the microcarrier, and any other site capable of ligation with the molecular identifier.

6. The method for high-resolution spatial omic detection of a tissue sample according to claim 1, wherein a material of the slide in the device comprises any material usable for preparing a morphological structure; the slide comprises at least one microwell reaction chamber array; the microwell reaction chamber array comprises at least one microwell; and each microwell in the microwell reaction chamber array comprises at least one microcarrier.

7. The method for high-resolution spatial omic detection of a tissue sample according to claim 1, wherein a shape of the microwell comprises regular and irregular three-dimensional morphological structures, and an internal volume of the microwell reaction chamber ranges from 0.1 fm³ to 1 cm³.

8. The method for high-resolution spatial omic detection of a tissue sample according to claim 1, wherein an approach of transferring molecular identifiers to the microwell reaction chamber array comprises direct and indirect adding approaches; ways of ligation of the molecular identifier with the microcarrier comprise but are not limited to physical modification, chemical modification, and biological modification.

9. The method for high-resolution spatial omic detection of a tissue sample according to claim 1, wherein a number of channels of the microchannels arranged in parallel is one or more.

10. The method for high-resolution spatial omic detection of a tissue sample according to claim 1, wherein both of a width of a channel and a spacing width between channels of the microchannels arranged in parallel range from 0.1 nm to 1000 μm.

11. The method for high-resolution spatial omic detection of a tissue sample according to claim 1, wherein the universal domain comprises:
i. a functional group modification site, a substance capable of binding to a microcarrier or a precursor capable of being activated to form a reactive functional group;
ii. a universal polymerase chain reaction (PCR) amplification starting end, capable of complementary binding with a universal primer and used for extension or amplification of a nucleic acid molecule; and
iii. a splicing domain, used for releasing a generated nucleic acid molecular identifier from the microcarrier.

12. The method for high-resolution spatial omic detection of a tissue sample according to claim 1, comprising the step of co-incubating the hybridized and complemented first and second nucleic acid molecular identifiers and the reaction mixture liquid such that the microcarrier generates the unique nucleic acid molecular identifier, wherein the unique nucleic acid molecular identifier comprises in the direction from 5′ to 3′: the universal domain, the first location domain, the ligation domain, the second location domain, the molecular marker, and the capture domain, or comprises: the universal domain, the first location domain, the second location domain, the molecular marker, and the capture domain; and the reaction mixture liquid preferably comprises any component capable of extending, amplifying, or ligating a nucleic acid sequence.

13. The method for high-resolution spatial omic detection of a tissue sample according to claim 1, wherein an arrangement of functional regions of the first nucleic acid molecular identifier, the second nucleic acid molecular identifier, and the unique nucleic acid molecular identifier comprises but is not limited to an order, positions, or contents listed in the present disclosure; and one or more functional sequences of the molecular identifiers are preferably arranged in any suitable order or contents.

14. The method for high-resolution spatial omic detection of a tissue sample according to claim 1, wherein the universal domain, the universal PCR amplification starting end, the first location domain, the second location domain, the ligation domain, the ligation domain complementary region, the molecular marker, the capture domain precursor, and the capture domain sequences are each at least one nucleotide long.

15. The method for high-resolution spatial omic detection of a tissue sample according to claim 1, wherein a way of embedding the tissue sample into microwells comprises introducing the tissue sample into the microwells by using any external force or by means of intrinsic properties of the microwell reaction chamber or by means of properties of a solid-phase or liquid-phase compound; a class of the solid-phase or liquid-phase compound comprises any compound capable of introducing a tissue slice into the microwells; a class of the porous membrane comprises a porous membrane of any material; and a pore diameter of the porous membrane ranges from 0.1 nm to 100 mm.

16. The method for high-resolution spatial omic detection of a tissue sample according to claim 1, comprising a step of recovering from the microcarrier, creating a library for, and analyzing the unique nucleic acid molecular identifier, or a hybrid strand or a complementary double-stranded nucleic acid sequence generated by the unique nucleic acid molecular identifier and a captured nucleic acid, and any nucleic acid sequence obtained through conversion by the method, wherein the step is preferably performed on the microcarrier, or performed after recovering the unique nucleic acid molecular identifier with the captured nucleic acid information or the complementary double-stranded nucleic acid sequence thereof from the microcarrier.

17. The method for high-resolution spatial omic detection of a tissue sample according to claim 1, wherein when amplifying and creating a library for a target sequence nucleic acid with the captured sequence information, amplifying and library creating approaches comprise any known nucleic acid amplifying and library creating approaches.

18. The method for high-resolution spatial omic detection of a tissue sample according to claim 1, expanded to capture and analyze nucleic acid, protein, and polysaccharide molecules in tissue, cell, and virus samples.

19. The method for high-resolution spatial omic detection of a tissue sample according to claim 1, wherein the tissue sample is a tissue sample of any living body or a spatial structure of the living body.

20. The method for high-resolution spatial omic detection of a tissue sample according to claim 1, wherein the tissue sample is a tissue sample of any type or class; and the tissue sample also comprises any treated or untreated tissue sample.

21. The method for high-resolution spatial omic detection of a tissue sample according to claim 1, applied to obtain or retrieve exclusive or independent omic information of a single cell or a plurality of cells of any type.

22. The method for high-resolution spatial omic detection of a tissue sample according to claim 1, applied to conduct a spatial transcriptomic study on a tissue slice:
specifically comprising: introducing a solid-phase or liquid-phase compound into the microwell reaction chamber array in which the microcarriers are stored, attaching the tissue slice to a surface of the microwell array, embedding a tissue sample into microwells, covering a surface of the microwells with a porous membrane for preventing cross pollution between the spatial omic information of the tissue sample, and adding a tissue permeabilizing liquid to a surface of the porous membrane, wherein the microcarrier captures the mRNA of the tissue confined in the microwell by means of the capture domain of the unique nucleic acid molecular identifier, and the surface is cleaned; incubating a reverse transcription reaction mixture liquid in the microwell array, and extending and synthesizing a hybrid strand of which the omic information has been captured such that the captured mRNA and the unique nucleic acid molecular identifier form cDNA, followed by amplifying and creating a library for cDNA; recovering the nucleic acid sequence and analyzing the recovered nucleic acid sequence;
next, based on the information of the first location domain and the second location domain, causing the analyzed tissue or cell sample-derived transcriptomic information to correspond to spatial points of the tissue sample by position information, thereby obtaining spatial transcription information of the tissue sample.

23. The method for high-resolution spatial omic detection of a tissue sample according to claim 1, applied to any class of biological omic testing and analysis.
